SEQUENCE LISTING (14 sequences)

SEQ ID NO: 1 - protein, 892 aa, Artificial Sequence
  Synthetic Polypeptide
MACLHETRTPSPSFGGFVSTLSEASMRKLDPDTSDCTPEKDLTPTQCVLRDVVPLGGQGGGGPSPSPGGEPPPEPFANSVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

SEQ ID NO: 2 - protein, 242 aa, Artificial Sequence
  Synthetic Polypeptide
ILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDI

SEQ ID NO: 3 - protein, 116 aa, Artificial Sequence
  Synthetic Polypeptide
LSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTME

SEQ ID NO: 4 - protein, 22 aa, Artificial Sequence
  Synthetic Polypeptide
VKLHFEKIKSEGTCLHRLEEEL

SEQ ID NO: 5 - protein, 22 aa, Artificial Sequence
  Synthetic Polypeptide
LNALMLQLELKERELLRREQAL

SEQ ID NO: 6 - protein, 520 aa, Artificial Sequence
  Synthetic Polypeptide
MACLHETRTPSPSFGGFVSTLSEASMRKLDPDTSDCTPEKDLTPTQCVLRDVVPLGGQGGGGPSPSPGGEPPPEPFANSVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMAFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTME

SEQ ID NO: 7 - protein, 520 aa, Artificial Sequence
  Synthetic Polypeptide
MACLHETRTPSPSFGGFVSTLSEASMRKLDPDTSDCTPEKDLEPTQCVLRDVVPLGGQGGGGPSPSPGGEPPPEPFANSVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMAFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTME

SEQ ID NO: 8 - protein, 116 aa, Artificial Sequence
  Synthetic Polypeptide
LATPQETYFKSQAEWREEVKKHFEKIKSEGTCIHRLDEELIRRRREELRHALDIREHYERKLERANNLYMELSAIMLQLEMREKELIKREQAVEKKYPGTYKRHPVRPIIHPNAME

SEQ ID NO: 9 - DNA, 2680 bp, Artificial Sequence
  Synthetic Polynucleotide
atggcttgcctccatgagacccgaacaccctctccttcctttgggggctttgtgtctaccctaagtgaggcatccatgcgcaagctggacccagacacttctgactgcactcccgagaaggacctgacgcctacccagtgtgtacttcgagatgtggtaccccttggtgggcagggtgggggagggcccagcccctccccaggtggagagccgccccctgagccttttgccaacagtgtcctgcagctacatgagcaggatgcagggggcccagggggagcagctgggtcacctgagagtcgggcatccagagttcgagctgacgaggtgcgactgcagtgccagagtggcagtggcttccttgagggcctctttggctgcctgcgccctgtctggaccatgattggcaaagcctactccactgagcacaagcagcagcaggaagacctttgggaggtcccctttgaggaaatcctggacctgcagtgggtgggctcaggggcccagggtgctgtcttcctggggcgcttccacggggaggaggtggctgtgaagaaggtgcgagacctcaaagaawaccgacatcaagcacttgcgaaagctgaagcaccccaacatcatcactttcaagggtgtgtgcacccaggctccctgctactgcatcctcatggagttctgcgcccagggccagctgtatgaggtactgcgggctggccgccctgtcaccccctccttactggttgactggtccatgggcatcgctggtggcatgaactacctgcacctgcacaagattatccacagggatctcaagtcacccaacatgctaatcacctacgacgatgtggtgaagatctcagattttggcacttccaaggagctgagtgacaagagcaccaagatgtcctttgcagggacagtagcctggatggcccctgaggtgatccgcaatgaacctgtgtctgagaaggtcgacatctggtcctttggcgtggtgctatgggaactgctgactggtgagatcccctacaaagacgtagattcctcagccattatctggggtgtgggaagcaacagtctccatctgcccgtgccctccagttgcccagatggtttcaagatcctgcttcgccagtgctggaatagcaaaccacgaaatcgcccatcattccgacagatcctgctgcatctggacattgcctcagctgatgtactctccacaccccaggagacttactttaagtcccaggcagagtggcgggaagaagtaaaactgcactttgaaaagattaagtcagaagggacctgtctgcaccgcctagaagaggaactggtgatgaggaggagggaggagctcagacacgccctggacatcagggagcactatgaaaggaagctggagagagccaacaacctgtatatggaacttaatgccctcatgttgcagctggaactcaaggagagggagctgctcaggcgagagcaagctttagagcggaggtgcccaggcctgctgaagccacacccttcccggggcctcctgcatggaaacacaatggagaagcttatcaagaagaggaatgtgccacagaagctgtcaccccatagcaaaaggccagatatcctcaagacggagtctttgctccctaaactagatgcagccctgagtggggtggggcttcctgggtgtcctaagggccccccctcaccaggacggagtcgccgtggcaagacccgtcaccgcaaggccagcgccaaggggagctgtggggacctgcctgggcttcgtacagctgtgccaccccatgaacctggaggaccaggaagcccagggggcctaggagggggaccctcagcctgggaggcctgccctcccgccctccgtgggcttcatcatgacctcctgctccgcaaaatgtcttcatcgtccccagacctgctgtcagcagcactagggtcccggggccggggggccacaggcggagctggggatcctggctcaccacctccggcccggggtgacaccccaccaagtgagggctcagcccctggctccaccagcccagattcacctgggggagccaaaggggaaccacctcctccagtagggcctggtgaaggtgtggggcttctgggaactggaagggaagggacctcaggccggggaggaagccgggctgggtcccagcacttgaccccagctgcactgctgtacagggctgccgtcacccgaagtcagaaacgtggcatctcatcggaagaggaggaaggagaggtagacagtgaagtagagctgacatcaagccagaggtggcctcagagcctgaacatgcgccagtcactatctaccttcagctcagagaatccatcagatggggaggaaggcacagctagtgaaccttcccccagtggcacacctgaagttggcagcaccaacactgatgagcggccagatgagcggtctgatgacatgtgctcccagggctcagaaatcccactggacccacctccttcagaggtcatccctggccctgaacccagctccctgcccattccacaccaggaacttctcagagagcggggccctcccaattctgaggactcagactgtgacagcactgaattggacaactccaacagcgttgatgccttgcggcccccagcttccctccctccatga

SEQ ID NO: 10 - DNA, 1563 bp, Artificial Sequence
  Synthetic Polynucleotide
atggcttgcctccatgagacccgaacaccctctccttcctttgggggctttgtgtctaccctaagtgaggcatccatgcgcaagctggacccagacacttctgactgcactcccgagaaggacctgacgcctacccagtgtgtacttcgagatgtggtaccccttggtgggcagggtgggggagggcccagcccctccccaggtggagagccgccccctgagccttttgccaacagtgtcctgcagctacatgagcaggatgcagggggcccagggggagcagctgggtcacctgagagtcgggcatccagagttcgagctgacgaggtgcgactgcagtgccagagtggcagtggcttccttgagggcctctttggctgcctgcgccctgtctggaccatgattggcaaagcctactccactgagcacaagcagcagcaggaagacctttgggaggtcccctttgaggaaatcctggacctgcagtgggtgggctcaggggcccagggtgctgtcttcctggggcgcttccacggggaggaggtggctgtgaagaaggtgcgagacctcaaagaaaccgacatcaagcacttgcgaaagctgaagcaccccaacatcatcactttcaagggtgtgtgcacccaggctccctgctactgcatcctcatggagttctgcgcccagggccagctgtatgaggtactgcgggctggccgccctgtcaccccctccttactggttgactggtccatgggcatcgctggtggcatgaactacctgcacctgcacaagattatccacagggatctcaagtcacccaacatgctaatcacctacgacgatgtggtgaagatctcagattttggcacttccaaggagctgagtgacaagagcaccaagatggcctttgcagggacagtagcctggatggcccctgaggtgatccgcaatgaacctgtgtctgagaaggtcgacatctggtcctttggcgtggtgctatgggaactgctgactggtgagatcccctacaaagacgtagattcctcagccattatctggggtgtgggaagcaacagtctccatctgcccgtgccctccagttgcccagatggtttcaagatcctgcttcgccagtgctggaatagcaaaccacgaaatcgcccatcattccgacagatcctgctgcatctggacattgcctcagctgatgtactctccacaccccaggagacttactttaagtcccaggcagagtggcgggaagaagtaaaactgcactttgaaaagattaagtcagaagggacctgtctgcaccgcctagaagaggaactggtgatgaggaggagggaggagctcagacacgccctggacatcagggagcactatgaaaggaagctggagagagccaacaacctgtatatggaacttaatgccctcatgttgcagctggaactcaaggagagggagctgctcaggcgagagcaagctttagagcggaggtgcccaggcctgctgaagccacacccttcccggggcctcctgcatggaaacacaatggagtag

SEQ ID NO: 11 - DNA, 1563 bp, Artificial Sequence
  Synthetic Polynucleotide
atggcttgcctccatgagacccgaacaccctctccttcctttgggggctttgtgtctaccctaagtgaggcatccatgcgcaagctggacccagacacttctgactgcactcccgagaaggacctggagcctacccagtgtgtacttcgagatgtggtaccccttggtgggcagggtgggggagggcccagcccctccccaggtggagagccgccccctgagccttttgccaacagtgtcctgcagctacatgagcaggatgcagggggcccagggggagcagctgggtcacctgagagtcgggcatccagagttcgagctgacgaggtgcgactgcagtgccagagtggcagtggcttccttgagggcctctttggctgcctgcgccctgtctggaccatgattggcaaagcctactccactgagcacaagcagcagcaggaagacctttgggaggtcccctttgaggaaatcctggacctgcagtgggtgggctcaggggcccagggtgctgtcttcctggggcgcttccacggggaggaggtggctgtgaagaaggtgcgagacctcaaagaaaccgacatcaagcacttgcgaaagctgaagcaccccaacatcatcactttcaagggtgtgtgcacccaggctccctgctactgcatcctcatggagttctgcgcccagggccagctgtatgaggtactgcgggctggccgccctgtcaccccctccttactggttgactggtccatgggcatcgctggtggcatgaactacctgcacctgcacaagattatccacagggatctcaagtcacccaacatgctaatcacctacgacgatgtggtgaagatctcagattttggcacttccaaggagctgagtgacaagagcaccaagatggcctttgcagggacagtagcctggatggcccctgaggtgatccgcaatgaacctgtgtctgagaaggtcgacatctggtcctttggcgtggtgctatgggaactgctgactggtgagatcccctacaaagacgtagattcctcagccattatctggggtgtgggaagcaacagtctccatctgcccgtgccctccagttgcccagatggtttcaagatcctgcttcgccagtgctggaatagcaaaccacgaaatcgcccatcattccgacagatcctgctgcatctggacattgcctcagctgatgtactctccacaccccaggagacttactttaagtcccaggcagagtggcgggaagaagtaaaactgcactttgaaaagattaagtcagaagggacctgtctgcaccgcctagaagaggaactggtgatgaggaggagggaggagctcagacacgccctggacatcagggagcactatgaaaggaagctggagagagccaacaacctgtatatggaacttaatgccctcatgttgcagctggaactcaaggagagggagctgctcaggcgagagcaagctttagagcggaggtgcccaggcctgctgaagccacacccttcccggggcctcctgcatggaaacacaatggagtag

SEQ ID NO: 12 - DNA, 354 bp, Artificial Sequence
  Synthetic Polynucleotide
atgcttgccaccccacaagaaacttacttcaagtctcaggctgaatggagagaagaagtgaaaaaacattttgagaagatcaaaagtgaaggaacttgtatacaccggttagatgaagaactgattcgaaggcgcagagaagagctcaggcatgcgctggatattcgtgaacactatgagcggaagcttgagcgggcgaataatttatacatggaattgagtgccatcatgctgcagctagaaatgcgggagaaggagctcattaagcgtgagcaagcagtggaaaagaagtatcctgggacctacaaacgacaccctgttcgtcctatcatccatcccaatgccatggagtga

SEQ ID NO: 13 - protein, 859 aa, Artificial Sequence
  Synthetic Polypeptide
MACLHETRTPSPSFGGFVSTLSEASMRKLDPDTSDCTPEKDLTPTHVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

SEQ ID NO: 14 - DNA, 591 bp, Artificial Sequence
  Synthetic Polynucleotide
atcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

(19) United States
(12) Patent Application Publication
(10) Pub. No.: US 2024/0216537 A1
(43) Pub. Date: Jul. 4, 2024

(54) GENE THERAPY FOR NEUROPROTECTION

(71) Applicant: THE REGENTS OF THE UNIVERSITY OF CALIFORNIA, Oakland, CA (US)

(72) Inventors: Derek S. WELSBIE, La Jolla, CA (US); Mai T. VU, La Jolla, CA (US); Cassidy D. LEE, La Jolla, CA (US); Amit K. PATEL, La Jolla, CA (US)

(21) Appl. No.: 18/554,627

(22) PCT Filed: Apr. 11, 2022
(86) PCT No.: PCT/US2022/024310

§ 371 Date: Oct. 9, 2023

Related U.S. Application Data
(60) Provisional application No. 63/173,904, filed on Apr. 12, 2021.
(60) Provisional application No. 63/190,132, filed on May 18, 2021.

(51) Int. Cl. A61K 48/00; A61K 38/00; A61P 27/06; C12N 5/0793; C12N 9/12; C12N 15/86

(52) U.S. Cl. CPC A61K 48/005; A61P 27/06; C12N 5/0619; C12N 9/12; C12N 15/86; A61K 38/00; C12N 2750/14143; C12Y 207/11025

(57) ABSTRACT

Provided herein, inter alia, are methods and compounds for treating and preventing neural cell death, e.g., retinal ganglion cell death. Also provided herein, inter alia, are methods and compounds for treating and preventing nerve degeneration. The methods and compounds provided herein may treat or prevent glaucoma.

### CROSS-REFERENCE TO RELATED APPLICATIONS

This application claims priority to U.S. Provisional Application No. 63/190,132, filed May 18, 2021 and U.S. Provisional Applications No. 63/173,904, filed Apr. 12, 2021, each of which is incorporated by reference for all purposes.

### BACKGROUND

Dual leucine zipper kinase (DLK) and leucine zipper kinase (LZK) play a role in neural kinase signaling pathways, including in axon degeneration and cell death signaling in neuronal cells, including ganglion cells such as in retinal ganglion cells (RGCs) (Welsbie et. al., Neuron 94:1142-54, 2017). Inhibition and knockdown of DLK has been demonstrated to have neuroprotective effects in cellular and animal models of Alzheimer's disease, glaucoma, Parkinson's disease and other neurodegenerative conditions. (Ferratis et al., 2013, Dual leucine zipper kinase as a therapeutic target for neurodegenerative conditions Future Medicinal Chemistry 5:16). DLK mutants with dominant negative DLK (dnDLK) activity have been described (e.g., Chen et al., J Neurosci 28:672-80, 2008).

### BRIEF SUMMARY

In one aspect, provided herein is a nucleic acid encoding a dominant negative Dual Leucine Zipper Kinase (dnDLK) polypeptide comprising an amino acid segment with a sequence with at least 95% identity to region 1-520 of SEQ ID NO:1, wherein the polypeptide comprises at least one mutation, as determined with reference to SEQ ID NO:1, wherein the mutation is selected from a substitution at position 43, a substitution at position 302, and a substitution at position 516. In some embodiments, the dnDLK polypeptide comprises a substitution at position 302, wherein the substitution is any amino acid other than threonine. In some embodiments, the substitution at position 302 is S302A. In some embodiments, the dnDLK polypeptide comprises a substitution at position 43. In some embodiments, the substitution at position 43 is E or D. in some embodiments, the dnDLK polypeptide comprises a substitutions T43E and S302A. In some embodiments, the dnDLK polypeptide further comprises a substitution at position 185, such as K185A. In some embodiments, the dnDLK polypeptide comprises a substitution at position 516. In some embodiments, the substitution at position 516 is G516V. In some embodiments, the dnDLK polypeptide further comprises a substitution at position 424 or 426. In some embodiments, the dnDLK polypeptide further comprises a substitution at position 431, 438, 440, 445, 447, 486, 491, or 493. In some embodiments, the nucleic acid encodes a dominant negative Dual Leucine Zipper Kinase (dnDLK) polypeptide comprising the amino acid sequence of SEQ ID NO: 6 or SEQ ID NO:7.

In another aspect, the disclosure provides an isolated nucleic acid encoding a dominant negative dual leucine zipper (dnDLK) polypeptide comprising an amino acid sequence having at least 95% identity to region 158-520 of SEQ ID NO:1, wherein the polypeptide comprises at least one mutation, as determined with reference to SEQ ID NO:1, wherein the mutation is selected from a substitution at position 302 and a substitution at position 516. In some embodiments, the dnDLK polypeptide comprises a substitution at position S302, wherein the substitution is any amino acid other than threonine. In some embodiments the substitution is S302A. In some embodiments, the dnDLK polypeptide comprises a substitution at position 516, e.g., G516V. In some embodiments, the the dnDLK polypeptide further comprises a substitution at position 185, e.g., K185A. In some embodiments, the dnDLK polypeptide also comprises a substitution at position 424 and/or 426; and in further embodiments, comprises a substitution at position 431, 438, 440, 445, 447, 486, 491, or 493.

In a further aspect, the disclosure provides an isolated nucleic acid encoding a leucine zipper polypeptide that inhibits homodimerization and DLK heterodimerizaton with (LZK), wherein the polypeptide comprises an amino acid sequence having at least 95% identity to SEQ ID NO:8. In some embodiments, the polypeptide is fewer than 150 amino acids in length.

In another aspect, the disclosure provides a vector comprising a nucleic acid as described herein, e.g., in the preceding paragraphs of this section. In some embodiments, the vector is a viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAV9, AAV10, AAV11-derived or pseudotyped AAV-derived vector. In some embodiments, the vector is AAV2.7m8. In some embodiments, the vector further comprises a Woodchuck Hepatitis Virus Posttranscriptional Regulatory Element (WPRE).

In a further aspect, the disclosure provides a host cell comprising a nucleic acid encoding a dominant negative polypeptide as described herein, or a vector comprising the nucleic acid. In some embodiments, the neuron is a retinal ganglion. In some embodiments, the host cell is a mammalian cell. In some embodiments, the host cell is a human cell.

In an additional aspect, the disclosure provide a method of inhibiting neuronal cell death, the method comprising introducing a nucleic acid encoding a dominant negative polypeptide as described herein, or a vector comprising the nucleic acid, into a neural cell. In some embodiments, the neural cell is ex vivo. In some embodiments, the neural cell is in vivo. In some embodiments, the neural cell is an ophthalmic neuron. In some embodiments, the ophthalmic neuron is a retinal ganglion. In some embodiments, the neural cell is a photoreceptor cell. In some embodiments, the neural cell is mammalian. In some embodiments, the neural cell is a human neural cell.

in another aspect, the disclosure provides a method of treating or preventing neural cell death in a subject in need thereof, the method comprising administering a nucleic acid encoding a dominant negative polypeptide as described herein, or a vector comprising the nucleic acid, to the subject. In some embodiments, the subject has glaucoma, age-related macular degeneration, choroidal neovascularization (CNV), myopia-associated CNV, diabetic retinopathy, macular oedema, or retinal vein occlusion. In some embodiments, the subject has an inherited retinal disease. In some embodiments, the inherited retinal disease is retinitis pigmentosa.

In another aspect, the invention provides a polypeptide encoded by a nucleic acid encoding a dominant negative polypeptide as described herein, e.g., in the preceding paragraphs in this section.

### DESCRIPTION OF DRAWINGS

FIGS. 1A and 1B. Novel DLK/LZK-based transgenes robustly improve survival of retinal ganglion cells compared to existing approaches. (A) Immunopanned RGCs were transduced with lentivirus expressing the various transgenes and cultured in the presence of a DLK/LZK inhibitor to allow time for completion of the viral lifecycle and transgene expression. After 3-4 days, the inhibitor was withdrawn and the amount of survival was measured after three days, in the presence of a novel transgene (S302A, Delta C-term DLK) or a known dominant-negative (K185A) full-length DLK. (B) Survival was measured in presence of an LZK leucine zipper (LZ) ora known dominant-negative DLK LZ. In both cases, the novel transgenes were both more efficacious (approaching the survival of control cells that remained in the presence of the inhibitor) and more potent.

FIG. 2 provides data illustrating the activity of various mutant DLK polypeptides.

FIG. 3 provides data illustrating average axon health score of axons in a rat model of glaucoma following administration of dnDLK AAV or control AAV (left panel) and average percentage of optic nerved head degeneration as assessed by evaluating axon degeneration (right panel).

### DETAILED DESCRIPTION

While various embodiments and aspects of the present invention are shown and described herein, it will be understood by persons skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Practice of the invention involves common molecular biology techniques known in the art. Such techniques are described, for example, in a number of manuals such as Sambrook & Russell, Molecular Cloning, A Laboratory Manual (4th Ed, 2012); and Current Protocols in Molecular Biology (Ausubel, et al., John Wiley and Sons, New York, 1987-Volume 133, December 2020).

The following definitions are provided to facilitate understanding of certain terms used frequently herein and are not meant to limit the scope of the present disclosure.

### 1. Terminology

A “dual leucine zipper kinase” (“DLK”) is a mitogen-activated protein kinase kinase kinase (MAP3K) mixed lineage kinase family member and member of the ser/thr kinase superfamily. It plays a role in neural kinase signaling pathways, including neuronal cell death signaling. DLK comprises an N-terminal domain, a catalytic domain, a leucine zipper domain comprising two leucine zippers, and a C-terminal domain. Illustrative human DLK polypeptide sequences are available under UniProtKB entry Q12852. Human DLK is encoded by the mitogen-activated protein kinase kinase kinase 12 gene (MAP3K12), which is cytogenetically localized to chromosome region 12q13.13. A “human DLK” refers to any allelic form encoded by a human MAP3K12 gene. Two protein isoforms have been identified: isoform 1 as designated in UniProtKB entry Q12852 (sequence provided in SEQ ID NO:13) and isoform 2 as designated in UniProtKB entry Q12852 (sequence provided in SEQ ID NO:1; see also, GenBank accession number XM_011538725, protein sequence accession number XP_011537027). As used herein, “DLK” refers to human and non-human DLK polypeptides and polynucleotides, e.g., mammalian DLK sequences, such as mouse or rat DLK sequences. The following schematic depicts illustrative fragments that contain the N-terminal domain, kinase domain, leucine zipper and C-terminal domain. The leucine zipper domain of human DLK SEQ ID NO:1 corresponds to about amino acids 420-500 and can also be defined as extending for seven more amino acids based on the leucine zipper motif of having a leucine in the fourth position of the heptad. See, also, Nihalani et al., J. Biol. Chem. 275: 7273-7279, 2000)

| N-term | Kinase domain | LZ | LZ | C-terminus |
|---|---|---|---|---|
| 1 | 135 | 404 | 520 | 688 |

Leucine zipper kinase (LZK) is an MAP3K family member structurally related to DLK that also plays a role in neural signaling pathways, including neuronal cell death. LZK comprises an N-terminal domain, a catalytic domain (“kinase domain”), a leucine zipper domain comprising two leucine zippers, and a C-terminal domain. Illustrative human LZK polypeptide sequences are available under UniProtKB entry O43283. Human LZK is encoded by the mitogen-activated protein kinase kinase kinase 13 gene (MAP3K13), which is cytogenetically localized to chromosome region 3q27.2. A “human LZK” refers to any allelic form encoded by a human MAP3K13 gene. Five isoforms of LZK have been identified. Isoform 1 (O42283-1) is designated in the UniProt entry as the canonical isoform. The amino acid sequence of the leucine zipper domain of isoform 1 is provided in SEQ ID NO:8. As used herein, “LZK” refers to human and non-human LZK polypeptides and polynucleotides, e.g., mammalian LZK sequences, such as mouse and rat DLK sequences.

The term “dominant negative,” refers to a dominant negative DLK protein variant (dnDLK) or a LZK leucine zipper domain polypeptide (dnLZ) or variant thereof that is neuroprotective when expressed in neurons in which endogenous wildtype DLK is expressed. Without intending to be bound by a particular mechanism, dnDLK may inhibit DLK homodimerization.

A “variant” or “mutant,” in the context of a polypeptide sequence typically has at least 80% identity, or at least 85% identity to a reference amino acid sequence. In some embodiments, a variant polypeptide has at least 90% identity, or at least 91%, 92%, 93%, or 94% identity to a reference amino acid sequence. In some embodiments, a variant polypeptide has at least 95% identity, or at least 96%, 97%, 98%, or 99% identity to a reference amino acid sequence. The term “variant” also applies to nucleotide sequences, which, for example may have at least 70%, at least 75%, at least 80%, at least 85%, at least 90%, or at least 95%, or greater to a reference sequence.

The term “introduced into” a cell in the context of genetically modifying a cell to express a polypeptide refers to contacting the cells with a polynucleotide encoding the polypeptide under conditions in which the polynucleotide enters the cell and is expressed to produce protein. In relation to a protein, such as a dnDLK, the term “introduced into a cell(s)” includes for example and not limitation, transduction of the cell of a viral vector comprising a nucleic acid encoding the dnDLK, and expression of the encoded protein.

The terms “identical” or “percent identity,” in the context of two or more polypeptide or polynucleotide sequences, refer to two or more sequences or subsequences that are the same or have a specified percentage of amino acids or nucleotides that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region as measure by manual alignment and visual inspection or using a BLAST or BLAST 2.0, which are described in Altschul et al. (1990) J. Mol. Biol. 215: 403-410 and Altschul et al. (1977) Nucleic Acids Res. 25: 3389-3402, respectively, comparison algorithm (for nucleotide sequences) with default parameters; or BLASTP with default parameters for amino acid sequences. Software for BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 11, an expect threshold of 0.05, M=2, N=−3, and a comparison of both strands. For amino acid sequences, the BLASTP program uses as defaults a word size (W) of 6, an expect threshold of 0.05, and the BLOSUM62 scoring matrix (see Henikoff & Henikoff, Proc. Natl. Acad. Sci. USA 89:10915 (1989)).

The terms “corresponding to,” “determined with reference to,” or “numbered with reference to” when used in the context of the identification of a given amino acid residue in a polypeptide sequence of interest, refers to the position of the residue in a specified reference amino acid sequence when the polypeptide sequence of interest is maximally aligned and compared to the reference sequence. Thus, for example, an amino acid residue in a DLK polypeptide variant “corresponds to” an amino acid in the DLK sequence SEQ ID NO:1 when the residue in the variant aligns with the amino acid in SEQ ID NO:1 when the variant polypeptide sequence is optimally aligned to SEQ ID NO:1. The polypeptide that is aligned to the reference sequence need not be the same length as the reference sequence. Similarly, “corresponding to,” “determined with reference to,” or “numbered with reference to” when used in the context of the identification of a given nucleotide in a nucleic acid sequence of interest, refers to the position of the nucleotide in a specified polynucleotide reference sequence when the nucleic acid sequence of interest is maximally aligned and compared to the reference sequence.

A “substitution,” as used herein, denotes the replacement of one or more amino acids or nucleotides by different amino acids or nucleotides, respectively.

A “conservative” substitution, as used herein, refers to a substitution of an amino acid such that charge, polarity, hydropathy (hydrophobic, neutral, or hydrophilic), and/or size of the side group chain is maintained. Illustrative sets of amino acids that may be substituted for one another include (i) positively-charged amino acids Lys and Arg; and His at pH of about 6; (ii) negatively charged amino acids Glu and Asp; (iii) aromatic amino acids Phe, Tyr and Trp; (iv) aliphatic hydrophobic amino acids Ala, Val, Leu and Ile; and hydrophobic amino acid Met; (v) non-polar amino acids Ala, Val, Leu, lie, Pro, Phe, Trp, and Met; (vi) small polar uncharged amino acids such as Ser, Thr, and Asn; (vii) neutral hydrophilic amino acids Cys, Ser, Thr, Asn, Gln; (viii) small hydrophobic or neutral amino acids Gly, Ala, and Pro; (ix) amide-comprising amino acids Asn and Gln; and (x) branched amino acids Thr, Val, and Ile. In some embodiments, a conservative substitution may be based on size, for example, a small amino acid may be substituted with another small amino acid, such as Gly or Ala. In some embodiments, a hydroxyl-containing amino acid (Ser, Thr, or Tyr) may be substituted with an alternative hydroxyl-containing amino acid. Reference to the charge of an amino acid in this paragraph refers to the charge at pH 6-7.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. Nucleic acid molecules may be modified chemically or biochemically or may contain non-natural or derivatized nucleotide bases, as will be readily appreciated by those of skill in the art. The above term is also intended to include any topological conformation, including single-stranded or double-stranded forms. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The terms “protein” and “polypeptide” are used interchangeably unless indicated otherwise.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “vector” as used herein with respect to expression of dominant negative proteins or LZ domain polypeptide understood to refer to a recombinant nucleic acid construct that comprises a transgene that encodes a dnDLK or LZ polypeptide to be expressed in the host cell.

The term “viral vector” refers to a modified virus used to deliver transgenes into a host cell.

As used herein the term, “transgene” refers to a recombinant polynucleotide construct that can be introduced into a cell using a gene therapy vector, to result in expression in the cell of one or more proteins. In the context of the present disclosure, a transgene may include regulatory sequences controlling expression of the encoded protein(s) (for example, one or more of promoters, enhancers, terminator sequences, polyadenylation sequences, and the like), mRNA stability sequences (e.g. Woodchuck Hepatitis Virus Posttranscriptional Regulatory Element; WPRE), sequences that allow for internal ribosome entry sites (IRES) of bicistronic mRNA, sequences necessary for episome maintenance (e.g., ITRs and LTRs), sequences that avoid or inhibit viral recognition by Toll-like or RIG-like receptors (e.g. TLR-7, -8, -9, M DA-5, RIG-1 and/or DAI) and/or sequences necessary for transduction into cells.

As used herein, the terms “promoter” and “enhancer promoter” refer to a DNA sequence capable of controlling (e.g., increasing) the expression of a coding sequence or functional RNA. A promoter may include a minimal promoter (a short DNA sequence comprised of a TATA-box and other sequences that serve to specify the site of transcription initiation). An enhancer sequence (e.g., an upstream enhancer sequence) is a regulatory element that can interact with a promoter to control (e.g., increase) the expression of a coding sequence or functional RNA. As used herein, reference to a “promoter” may include an enhancer sequence. An enhancer does not need to be contiguous with a promoter or coding sequence with which it interacts.

Promoters, enhancers and other regulatory sequences are “operably linked” to a protein or RNA-encoding polynucleotide sequence when they affect to the expression or stability of the encoded mRNA or protein.

The terms “subject”, “patient” or “individual” are used herein interchangeably to refer to any mammal, including, but not limited to, a human. In some embodiments, the subject may be a non-human primate (e.g., a monkey, chimpanzee), a horse, a cow, a sheep, a pig, a goat, a dog, a cat, a mouse, a rat, a guinea pig, or any other mammal. In some embodiments, the subject”, “patient” or “individual” is a human.

### 2. Dominant Negative Polypeptide with Neuroprotective Activity

### 2.1 Dominant Negative DLK Sequences

In one aspect, the disclosure provides dominant negative DLK (dnDLK) polypeptides, and polynucleotides that encode dnDLK polypeptides. dnDLK are neuroprotective when introduced into a neuronal cell, such as a ganglion cell, e.g., a retinal ganglion cell. The term “neuroprotective” in this context refers to the ability of a dnDLK to inhibit neuronal cell death, e.g., by at least 10%, 20%, 30%, 40%, 50%, 60%, 70%, 80%, or 90%, or greater, in a population of neuronal cells engineered as described herein to express the dnDLK polypeptide compared to a control neuronal cell population that does not express a dnDLK polypeptide. For illustration and not limitation, neuroprotective activity can be measured using methods described in Section 5, below. Other assays for neuroprotection include image-based live/dead cell counting or reporter immunofluorescence, such as phosphorylated JUN.

Human DLK exists in two isoforms. SEQ ID NO:1 is the sequence of the isoform designated in Uniprot entry 12852 as “isoform 2,” and is used herein as a reference sequence for the description of residue positions. It will be understood that mutations described herein in relation to SEQ ID NO:1 can be introduced into SEQ ID NO:1 and/or SEQ ID NO:13. The isoforms differ from each other at residue 46 of the reference sequence. Uniprot Q12852 entry Isoform 1 (SEQ ID NO:13) comprises a histidine at position 46. This H residue is replaced by the sequence QCVLRDVVPLGGQGGGGPSPSPGGEPPPEPFANS in Q12852 isoform 2 (SEQ ID NO:1), which leads to a polypeptide that is 33 amino acids longer than the polypeptide sequence shown in SEQ ID NO:13, but which is otherwise identical in sequence.

| | | UniProt Designation |
|---|---|---|
| SEQ ID NO: 1 | Isoform #2 (892 aa) | Q12852-2 |
| SEQ ID NO: 13 | Isoform #1 (859 aa) | Q12852-1 |

In some embodiments, a dnDLK polypeptide comprises a DLK catalytic region having at least 80%, or at least 85% amino acid sequence identity to SEQ ID NO:2 and a leucine zipper domain having at least 80%, or at least 85% identity to SEQ ID NO:3, where the dnDLK comprises a mutation at position 302 and/or 516, as determined with reference to SEQ ID NO:1, in which the residue is substituted relative to the residue present at that position in SEQ ID NO:1. In some embodiments, the dnDLK polypeptide is 890 amino acids or fewer in length, 850 amino acids or fewer in length, 750 amino acids or fewer in length, 650 amino acids or fewer in length, 550 amino acids or fewer in length, 450 amino acid or fewer in length, or 400, 390, 380, 370, or 360, or fewer amino acids in length. For example, with respect to the full-length sequence in SEQ ID NO:1, in some embodiments, over 300 amino acids, or over 350 amino acids, or over 370 amino acids can be deleted from the C-terminus of SEQ ID NO:1. In some embodiments, 10, 20, 30, 40, 50, 60, or more amino acid can be deleted from the N-terminus of SEQ ID NO:1. In some embodiments, the dnDLK polypeptide comprises a DLK catalytic region having at least 90%, at least 91%, at least 92%, at least 93%, or at least 94% identity to SEQ ID NO:2 and a leucine zipper domain having a at least 90%, at least 91%, at least 92%, at least 93%, or at least 94% identity to SEQ ID NO:3 and comprises a mutation at position 302 and/or 516. In some embodiments, the dnDLK polypeptide comprises a DLK catalytic region having at least 95%, at least 96%, at least 97%, at least 98%, or at least 99% identity to SEQ ID NO:2 and a leucine zipper domain having a at least having at least 95%, at least 96%, at least 97%, at least 98%, or at least 99% identity to SEQ ID NO:3. In some embodiments, the dnDLK polypeptide comprises a substitution at position 302, wherein the residue at position 302 is substituted with any amino acid other than threonine. In some embodiments, the dnDLK polypeptide comprises A at position 302. In some embodiments, the dnDLK comprises a residue G, V, L, or I at position 302. In some embodiments, the dnDLK polypeptide comprises a conservative substitution, other than threonine, for the S at position 302, e.g., N or Q. In some embodiments, the DLK polypeptide comprises a substitution at position 516, e.g., a dnDLK polypeptide comprises any amino acid residue other than G. In some embodiments, a dnDLK polypeptide comprises V at position 516. In some embodiments, a dnDLK comprises an I or L at position 516. In some embodiments, a dnDLK polypeptide comprises a substitution at position 302 and a substitution at position 516 as described herein, e.g., in this paragraph. In some embodiments, a dnDLK polypeptide further comprises a substitution at position 185 as determined with reference to SEQ ID NO:1. In some embodiments, the dnDLK polypeptide comprises A at position 185. In some embodiments, the dnDLK polypeptide comprises G, V, L, or I at position 185. In some embodiments, a dnDLK polypeptide comprises a substitution at position 302 and/or 516 and additionally comprises a substitution at position 424 and/or 426, as determined with reference to SEQ ID NO:1; and optionally, when the polypeptide comprises a substitution at position 424 and/or position 426, a substitution at position 431, 438, 440, 445, 447, 486, 491, or 493. In some embodiments, the dnDLK polypeptide comprises D or E at position 424 and/or D or E at position 426. In some embodiments, the dnDLK polypeptide further comprises one or more of the following: R at position 431, D or E at position 438, D or E at position 440, D or E at position 445, D or E at position 447, R at position 486, D or E at position 491; and D or E at position 493.

In some embodiments, the dnDLK polypeptide comprises a DLK catalytic region having at least 90%, at least 91%, at least 92%, at least 93%, or at least 94% identity to SEQ ID NO:2 and a leucine zipper domain having at least 90%, at least 91%, at least 92%, at least 93%, or at least 94% identity to SEQ ID NO:3; or to a fragment of the leucine zipper domain of at least 95 contiguous amino acids comprising the two leucine zippers, where the dnDLK comprises any amino acid other than S or T at position 302, which position is determined with references to SEQ NO:1. In some embodiments, the dnDLK polypeptide comprises a region having at least 96%, at least 97%, at least 98%, or at least 99% identity to SEQ ID NO:2 and a leucine zipper domain having at least 95%, at least 96%, at least 97%, at least 98%, or at least 99% identity to SEQ ID NO:3, to a fragment of the leucine zipper domain of at least 95 contiguous amino acids comprising the two leucine zippers, where the dnDLK comprises any amino acid other than S or T at position 302, which position is determined with references to SEQ NO:1. In some embodiments, the fragment of the leucine zipper domain comprises the two leucine zipper sequences, but lacks 1, 2, 3, 4, or 5 amino acids, or up to 10 amino acids, or up to 20 amino acids from the C-terminal region of the leucine zipper domain, but does not delete amino acids in leucine zipper 1 or leucine zipper 2. In some embodiments, the dnDLK polypeptide comprises A at position 302. In some embodiments, the dnDLK polypeptide comprises a residue such as G, V, L, or I at position 302. In some embodiments, the dnDLK polypeptide comprises Q or N at position 302. In some embodiments, the dnDLK polypeptide comprises the N-terminal domain (region of SEQ ID NO:1 from position 2 to the first residue of the catalytic domain in SEQ ID NO:1); or a fragment thereof. In some embodiments, the dnDLK comprises a D or E at position 43. In some embodiments, the dnDLK polypeptide comprises E at position 43.

As discussed in SECTION 5.2.1 below, introducing a substitution mutation at residue 302 (e.g., S302A) results in a dnDNK with greater neuroprotective activity than the DLK K185A variant. As discussed in SECTION 5.2.6 below, introducing a substitution mutation at residue 516 (e.g., G516V) results in a dnDNK. As discussed in SECTION 5.2.2 below, deletion of >350 residues, illustrated in SECTION 5.2.2 by deletion of 362 residues, from the C terminus of a dnDLK does not negatively affect the protective activity of the dnDLK. For example, as discussed in SECTION 5.2.3, deletion of the C-terminal region from the S302A dnDLK does not reduce the protective activity of the mutant and in some embodiments, enhances protective activity.

In some embodiments, a dnDLK polypeptide comprises a region having at least 80%, or at least 85% amino acid sequence identity to amino acids 2-520 of SEQ ID NO:1, wherein the dnDLK polypeptide comprises one or more mutations at position 43, 302, or 516, as determined with reference to SEQ ID NO:1, in which the residue is substituted relative to the residue at that position in SEQ ID NO:1. In some embodiments, a dnDLK polypeptide comprises a region having at least 90%, at least 91%, at least 92%, at least 93%, or at least 94% identity to amino acids 1-520 of SEQ ID NO:1. In some embodiments, a dnDLK polypeptide comprises a region having at least 95%, at least 96%, at least 97%, at least 98%, or at least 99% identity to amino acids 1-520 of SEQ ID NO:1. In some embodiments, a dnDLK polypeptide comprises a region having at least 90%, at least 91%, at least 92%, at least 93%, or at least 94% identity to a 300 amino acid region of amino acids 1-520 of SEQ ID NO:1 and comprises one or more mutations at position 43, 302, or 516, as determined with reference to SEQ ID NO:1 and as further provided below. In some embodiments, a dnDLK polypeptide comprises a region having at least 95%, at least 96%, at least 97%, at least 98%, or at least 99% identity to a 300 amino acid region of amino acids 1-520 of SEQ ID NO:1 and comprises one or more mutations at position 43, 302, or 516, as determined with reference to SEQ ID NO:1 and as further provided below. In some embodiments, the dnDLK polypeptide comprises the amino acid sequence of SEQ ID NO:6 or SEQ ID NO:7. In some embodiments, the dnDLK comprises a substitution at position 43 and at position 302. In some embodiments, a dnDLK polypeptide comprises a substitution at position 302 and position 516. In some embodiments, a dnDLK polypeptide comprises a substitution at position 43 and position 516. In some embodiments, the dnDLK polypeptide comprises mutations at each of positions 43, 302, and 516. In some embodiments, the dnDLK polypeptide comprises a substitution at position 302, wherein the residue at position 302 is substituted with any amino acid other than threonine. In some embodiments, the dnDLK polypeptide comprises A at position 302. In some embodiments, the dnDLK comprises a residue G, V, L, or I at position 302. In some embodiments, the dnDLK polypeptide comprises a conservative substitution, other than threonine, for the S at position 302, e.g., N or Q. In some embodiments, the dnDLK polypeptide comprises a D or E at position 43. In some embodiments, the dnDLK polypeptide comprises E at position 43. In some embodiments, the DLK polypeptide comprises a substitution at position 516, e.g., a dnDLK polypeptide comprises any amino acid residue other than G. In some embodiments, a dnDLK polypeptide comprises V at position 516, or I or L at position 516. In some embodiments, the dnDLk polypeptide comprises A at position 302 and E at position 43. In some embodiments, the dnDLK polypeptide is 850 amino acids or fewer in length, 750 amino acids or fewer in length, 650 amino acids or fewer in length, or 550 amino acids or fewer in length. In some embodiments, a dnDLK polypeptide comprises a substitution at position 43, 302 and/or 516 and further comprises a substitution at position 185 as determined with reference to SEQ ID NO:1. In some embodiments, a dnDLK polypeptide comprises a substitution at position 302 and position 185; a substitution at position 302 and 516; or a substitution at position 302, 516, and 185. In some embodiments, such a dnDLK polypeptide can further comprise a substitution at position 43. In some embodiments, the dnDLK polypeptide comprises A at position 185. In some embodiments, a dnDLK polypeptide comprising a substitution at position 43, position 302 and/or 516 (and optionally, a substitution at position 185); additionally comprises a substitution at position 424 and/or 426, as determined with reference to SEQ ID NO:1; and optionally, when the polypeptide comprises a substitution at position 424 and/or position 426, a substitution at position 431, 438, 440, 445, 447, 486, 491, or 493. In some embodiments, the dnDLK polypeptide comprises D or E at position 424 and/or D or E at position 426. In some embodiments, the dnDLK polypeptide further comprises one or more of the following: R at position 431, D or E at position 438, D or E at position 440, D or E at position 445, D or E at position 447, R at position 486, D or E at position 491; and D or E at position 493.

In some embodiments, the dnDLK polypeptide comprises an amino acid sequence having at least 90%, at least 91%, at least 92%, at least 93%, or at least 94% identity to SEQ ID NO:6; or at least 95%, at least 96%, at least 97%, at least 98%, or at least 99% identity to SEQ ID NO:6, wherein the dnDLK comprises any amino acid other than S or T at position 302 as numbered with reference to SEQ ID NO:1. In some embodiments, the dnDLK polypeptide comprises A at position 302. In some embodiments, the dnDLK polypeptide comprises a residue such as G, V, L, or I at position 302. In some embodiments, the dnDLK polypeptide comprises Q or N at position 302. In some embodiments, the dnDLK polypeptide comprises SEQ ID NO:6.

In some embodiments, the dnDLK polypeptide comprises an amino acid sequence having at least 90%, at least 91%, at least 92%, at least 93%, or at least 94% identity to SEQ ID NO:7; or at least 95%, at least 96%, at least 97%, at least 98%, or at least 99% identity to SEQ ID NO:7, wherein the dnDLK comprises any amino acid other than S or T at position 302 as numbered with reference to SEQ ID NO:1; and a substitution at position 43, as determined with reference to SEQ ID NO:1. In some embodiments, the dnDLK polypeptide comprises A at position 302. In some embodiments, the dnDLK polypeptide comprises a residue such as G, V, L, or I at position 302. In some embodiments, the dnDLK polypeptide comprises Q or N at position 302. In some embodiments, the dnDLK polypeptide comprises D or E at position 43. In some embodiments, the dnDLK polypeptide comprises E at position 43. In some embodiments, the dnDLK polypeptide comprises A at position 302 and E at position 43. In some embodiments, the dnDLK polypeptide comprises SEQ ID NO:7.

2.2 LZK Sequences that are Dominant Negative Inhibitors of DLK

As discussed in Section 5.2.4 we discovered that, surprisingly, the LZK leucine zipper domain (LZ polypeptide) had potent neuroprotective activity. Introduction of the LZ domain into neural cells, e.g., such as retinal ganglion cells, can have therapeutic benefit.

In one aspect, the disclosure further provides a polypeptide comprising a LZK leucine zipper domain that inhibits DLK homodimerization, where the polypeptide is neural protective when introduced into a neural cell, such as a ganglion cell, e.g., a retinal ganglion cell. In some embodiments, the polypeptide comprises an amino acid sequence having at least 95% identity, or at least 96%, 97%, 98%, or at least 99% identity to SEQ ID NO:8. In some embodiments, the LZK polypeptide comprises SEQ ID NO:8. In some embodiments, the LZK polypeptide is less than 150 or less than 120 amino acids in length. For the sake of convenience, such LZK polypeptides that inhibit DLK and polynucleotide sequences that encode the polypeptides are referred to herein as “dnDLK/LZK” sequences.

### 2.3 Variant Activity

A variant as described herein that inhibits neuronal cell death can be identified using a variety of assays. In some embodiments, a variant is evaluated for the ability to inhibit neuronal cell death.

An illustrative assay for evaluating inhibition of neuronal cell death is provided in the examples. This assay is described in the context of retinal ganglion cells. Briefly, a variant and controls, such as an empty vector control, are each introduced into a population of primary retinal ganglion cells, e.g. using lentiviral vectors expressing the dnDLK or dnDLK/LZK polypeptide. The cells are maintained using a DLK/LZK inhibitor, e.g., GNE 3511, to prevent cell death while the lentiviral dnDLK or dnDLK/LZK protein is expressed. On day 3-5 post transduction, when the dnDLK or dnDLK/LZK protein is expressed (indicated, e.g., by the mScarlet reporter expression), GNE 3511 is withdrawn from the primary RGCs to initiate cell death. Cells are assayed for survival 3 days post GNE withdrawal [in relative light units (RLU)] by adding a 50% volume of CellTiter-Glo (Promega G8462). Luminescence can be measured with a plate reader (Molecular Devices). The level of cell death can be compared to that of control cells treated with the GNE3511 (positive control) throughout the time period of the assay or can be determined relative to control cells in which GNE3511 is withdrawn (negative control). A variant dn DLK or variant dnDLK/LZK polypeptide as described herein typically has at least 20%, often at least 30%, or at least 40%, 50%, 60%, 70%, or greater protection compared to the level of cell survival obtained using a chemical DLK/LZK inhibitor, e.g., GNE 3511. In some embodiments, a variant dnDLK or variant dnDLK/LZK polypeptide as described herein has at least 80%, at least 90%, or greater protection compared to the control.

In some embodiments, alternative assays are employed to evaluate variant activity. Many such assays are known, including those based on flow cytometry, caspase activation, and measure of intracellular ATP. For example, suitable assays include image-based live/dead cell counting using cell inclusion dyes, such as Calcein AM to indicated live cells, or cell exclusion based dyes, such as Reddot1 to indicate dead or dying cells. Alternatively, immunofluorescence of phosphorylated JUN or other downstream DLK pathway members can be employed as markers of the efficacy of dnDLK or dnDLK/LZK constructs in inhibiting the DLK pathway

### 3. Polynucleotides/Expression Vectors

In another aspect, the present disclosure provides isolated nucleic acids encoding dominant negative polypeptides as described herein, expression vectors comprising the nucleic acids, and host cells comprising the expression vectors. Expression systems for producing dominant negative polypeptides include prokaryotic and eukaryotic systems, including for example, yeast, insect, avian, and mammalian expression systems. Illustrative expression systems are described in Sambrook, supra; and Ausbel, supra.

Non-limiting examples of suitable eukaryotic promoters (i.e., promoters functional in eukaryotic cells) for expression of a nucleic acid encoding a dominant negative polypeptide include a cytomegalovirus (CMV) immediate early gene promoter; herpes simplex virus thymidine kinase gene promoter; early and late SV40 promoters; long terminal repeats from retrovirus; human elongation factor-1 promoter; chicken beta-actin promoter; bovine growth hormone promoter; a hybrid construct comprising the CMV enhancer fused to the chicken beta-actin promoter, the first exon and first intron of the chicken beta-actin gene, and beta globin splice acceptor; murine stem cell virus promoter, phosphoglycerate kinase-1 locus promoter, and ubiquitin gene promoter. In some embodiments, the promoter is active in neural cells.

Expression vectors may also include other elements, for example, a ribosome binding site, a transcription terminator, and the like.

A nucleic acid encoding a dominant negative polypeptide of the disclosure can be introduced into a host cell using a variety of delivery methods, including packing into or on the surface of delivery vehicles for delivery to cells. Delivery vehicles include, but are not limited to, nanospheres, liposomes, quantum dots, nanoparticles, polyethylene glycol particles, hydrogels, and micelles. In some embodiments, targeting moieties can be used to enhance the preferential interaction of such vehicles with desired cell types or locations. In some embodiments, nucleic acid are introduced into host cells by viral infection, transfection, protoplast fusion, lipofection, electroporation nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

### 3.1 Gene Therapy Vectors

In certain embodiments, the present disclosure provides gene therapy vectors for genetically modifying cells, e.g., neuronal cells, either ex vivo or in vivo to express a dominant negative polypeptide as described herein. In some embodiments, the gene therapy vector is a plasmid vector, e.g., that can be delivered as naked DNA or complexed with a delivery formulation such as a lipid, liposome, nanoparticle or poloxamer.

In other embodiments, the gene therapy vector is a viral vector. Non-limiting examples of viral vectors include, but are limited to, lentivirus, adenovirus, adeno-associated virus, vaccinia virus, herpes simplex virus, pox poxviru, alphavirus, enterobirus, papovavirus, poliovirus, and other positive and negative stranded RNA viruses, viroids, and virusoids, or portions thereof.

A transgene may include regulatory sequence located upstream, downstream or within a coding sequence that influence RNA processing or stability, translation efficiency, or other aspects influencing efficient and stable expression of the dominant negative polypeptide. Examples of such regulatory sequence include promoter, enhance, polyadenylation sequences and the like.

In some embodiments, the viral vector further comprises a posttranscriptional regulatory elements such as a Woodchuck Hepatitis Virus Posttranscriptional Regulatory Element (WPRE) to enhance expression of transgenes delivered using a viral vector.

### 3.1.1 AAV Vectors

In some embodiments, a gene therapy vector is an adeno-associate virus vector (AAV). Any AAV serotype can be used for generating recombinant AAV for introducing a dominant negative polypeptide into neural cells, including, but not limited to, AAV1, AAV2, AAV3b, AAV4, AAV5, AAV6, AAV7, AAV8, AAV9, AAV10, AAV-11, AAV-12, AAV113, AAVrh.74, AAV2.7m8, AAV.ANC80, Anc80L65 and derivatives; AAVDJ, and combinations thereof. An extensive listing of illustrative AAV serotypes, including variants of naturally occurring AAV serotypes, is provided in U.S. Pat. No. 10,662,425.

The viral sequences include those sequences of AAV that are required in cis for replication and packaging (e.g., functional ITRs) of the DNA into a virion. In some embodiments, AAV vectors have one or more of the AAV WT genes deleted in whole or in part, but retain functional flanking ITR sequences. An AAV virion or AAV particle comprises an AAV capsid protein and an AAV vector. In some instances, the AAV is a hybrid or chimeric AAV, e.g., an AAV particle can comprise ITRs that are of a heterologous serotype in comparison with the capsid serotype (e.g., AAV2 ITRs with AAV5, AAV6, or AAV8 capsids). The AAV ITRs may be of any serotype suitable for a particular application. In some embodiments, an AAV serotype, such as AAV1, AAV2, AAV4, AAV5, AAV8, AAV9, or AAVrh8R is employed as a vector. In some embodiments, the vector is a derivative of an AAV2 serotype, e.g., AAV2.7m8.

Methods for using AAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery. For packaging a transgene into virions, the ITRs are the only AAV components required in cis in the same construct as the transgene. The cap and rep genes can be supplied in trans. The construction of recombinant AAV virions has also been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery. See also, Zolotukin et al., 2002, PRODUCTION AND PURIFICATION OF SEROTYPE 1, 2, AND 5 RECOMBINANT ADENO-ASSOCIATED VIRAL VECTORS” Methods 28:158-167.

In some embodiments, transgenes encoding dnDLK or dnDLK/LZK polypeptides are administered in combination with a transgene(s) encoding another protein with neuroprotective properties. As discussed above, deletion of C-terminal portions of dnDLK does not reduce the protective activity of the protein. The smaller size of a transgene encoding truncated dnDLK is advantageous in design of constructs encoding more than one polypeptide, e.g., more than one neuroprotective polypeptide. In some embodiments, a construct encoding a dnDLK or dnDLK/LZK polypeptide may further comprise a transgene encoding a polypeptide that lowers intraocular pressure, e.g., for glaucoma therapy, or a transgene encoding a protein that is associated with disease pathology. For example and not for limitation, a transgene encoding a dnDLK or dnDLK/LZK polypeptide and a dominant negative SARM1 may be used (see WO2019079572, “Dominant Negative Sarm1 Molecules As A Therapeutic Strategy For Neurodegenerative Diseases Or Disorders”; Geisler et al., 2019, “Gene therapy targeting SARM1 blocks pathological axon degeneration in mice” J Exp Med 216:294-303)). As another example, a recombinant AAV vector may express a dnDLK polypeptide or dnDLK/LZK polypeptide and a polypeptide such as insulin-like growth factor (see, e.g., Hung et al., 2007, “Gene transfer of insulin-like growth factor-I providing neuroprotection after spinal cord injury in rats.” J. Neurosurgery: Spine 6(1); Nishida et al., 2011, “Restorative effect of intracerebroventricular insulin-like growth factor-I gene therapy on motor performance in aging rats.” Neuroscience 177:195-206; Schwerdt et al., 2018, “Rejuvenating Effect of Long-Term Insulin-Like Growth Factor-I Gene Therapy in the Hypothalamus of Aged Rats with Dopaminergic Dysfunction.” Rejuv. Res. 21(2):102-108). As another example, a nicotinamide mononucleotide adenylyltransferase (NMNAT) polypeptide such as NMNAT1 (EC 2.7.7.1) protein may be expressed (see Babetto et al., 2010, “Targeting NMNAT1 to axons and synapses transforms its neuroprotective potency in vivo” J Neurosci 30(40):13291-304) with a dnDLK or dnDLK/LZK polypeptide. In some embodiments, the NMNAT polypeptide is an NMNAT polypeptide predominantly found in cytoplasm e.g., CytNMNAT1 (Sasaki et al, J. Neurosci 26:8484-8491, 2006), which is mutated in the nuclear localization signal; an NMNAT2 polypeptide or NMNAT2 polypeptide having a deletion in the subcellular targeting domain (Milde et al, Sci Rep. 3:2567, 2013); or an NMNAT3 polypeptide (Berger, et al, J Biol Chem 280(43), 36334-36341, 2005). As a further example, osteopontin may be expressed (see, e.g., Chen et al., 2011, “Osteopontin reduced hypoxia-ischemia neonatal brain injury by suppression of apoptosis in a rat pup model.” Stroke 42(3):764-769). As another example, glucagon-like peptide-1 may be expressed (Holscher, 2012, “Potential Role of Glucagon-Like Peptide-1 (GLP-1) in Neuroprotection.” CNS Drugs 26:871-882; Velmurugan et al., 2012, “Neuroprotective actions of Glucagon-like peptide-1 in differentiated human neuroprogenitor cells.” J. Neurochem. 123(6):919-931; WO2009039964A2 (“Use of glucagon-like peptide as a therapeutic agent”). As another example, brain derived neurotrophic factor may be expressed (see Osborne et al., 2018, “Neuroprotection of retinal ganglion cells by a novel gene therapy construct that achieves sustained enhancement of brain-derived neurotrophic factor/tropomyosin-related kinase receptor-B signaling.” Cell Death & Disease 9:1007). Further examples of proteins that can be expressed with a dnDLK or dnDLK/LZK polypeptide include the slow Wallerian degeneration polypeptide (Wld^{s)} (e.g., Conforti et al, Proc Natl Acad Sci USA 97:11377-82, 2000); dominant negative Rho-kinase (e.g., Amano et al, J. Biol. Chem 274:32418-24, 1999); or a matrix metalloprotease (MMP) such as MMP-3 (e.g., O'Callaghan et al, Hum. Mol. Genet. 26:1230-246, 2017) or MMP-1 (Borras et al, Gene ther. 23:438-49, 2016). In one approach, two or more genes may be encoded by the same RNA transcript (using a bicistronic expression cassette in a viral vector, e.g., AAV). For example, in one approach, expression of two genes is controlled by a bidirectional promoter (Vogl et al., 2018, “Engineered bidirectional promoters enable rapid multi-gene co-expression optimization” Nat Commun 9, 3589; Trinklein et al., 2004, “An Abundance of Bidirectional Promoters in the Human Genome” Genome Res. 14:62-66; Wang et al., 2006, “Suppression of experimental osteoarthritis by adenovirus-mediated double gene transfer” Chin Med J (Engl) 119: 1365-1373). In some embodiments, a transgene encoding a dnDLK or dnDLK/LZK polypeptide is administered in combination with guide RNAs to activate the promoter of an endogenous gene that encodes a polypeptide having neuroprotective properties.

In some embodiments, a transgene encoding a dnDLk or dnDLK/LZK polypeptide is administered in combination with a nucleic acid encoding a polypeptide involved in neuroprotection, such as an SCG10/STMN2, BCL-XL, or TRKB polypeptide. In some embodiments, the polypeptide is an aquaporin, CNTF polypeptide (with a signal sequence), BDNF, GDNF, or NGF polypeptide. In some embodiments, a complement inhibitor polypeptide is expressed. In some embodiments, a GLP-1R or GLP-1 polypeptide, or a CDKN2B-AS1 polypeptide is expressed. In some embodiments, a GLDN, CHL1, QPCT, TBX20, DGKG, TIMP2, EGR1, EOMES, JUNB, IGFBP2, OSTF1, FGF1, SEMA5A, ESRRG, KBTBD11, RAMP1, ETL4, PRKCQ, CTXN3, NDNF, MAN1A, SDK2, PRPH, SDK1, or IF127 polypeptide is expressed. In alternative embodiments, a transgene encoding a dnDLK or dnDLK/LZK polypeptide is administered in combination with CRISPR protein/guide RNAs to activate the promoter of an endogenous neuroprotective gene. For purposes of this paragraph, the gene nomenclature is also used to designate the polypeptide.

In some embodiments, a transgene encoding a dnDLK or dnDLK/LZK polypeptide is administered with an inhibitory agent that inhibits expression of a gene or activity of a product encoded by the gene in order to enhance neural protection. In some embodiments the inhibitory agent is a nucleic acid, e.g., antisense RNA or shRNAs that targets the gene to be inhibited. In some embodiments, the agent is CRISPR protein/gRNA protein to target a gene or the promoter of a gene to be inhibited. In some embodiments, an inhibitory protein is administered, e.g., an antibody (which as used here includes a nanobody, single chain immunoglobulin, or other antibody fragment that binds the target protein), a dominant-negative polypeptide and/or a protein that binds and targets the protein to be inhibited for proteasomal degradation. In some embodiments, the gene or gene product targeted for inhibition is involved in axonal or somal protection, e.g., MEKK4, MLK2/MAP3K10, PUMA/BBC3, SARM1, ROCK1, ROCK2, TAOK1, TAOK2, TAOK3, TNIK, MAP4K4, MINK1, GSK-3S, GSK-3a, MAP2K7/MKK7, MAP2K4/MKK4, PERK, CHOP, HSP90, SNRK, JNK1 (MAPK8), JNK2 (MAPK9), JNK3 (MAPK10), JUN, ATF2, MEF2A, SOX11, MST1/STK4, MST2/STK3, END1, or END2. In some embodiments, the gene or gene product targeted for inhibition is involved in glial interactions, e.g., C1q, TNFα, or IL-1α; or is a glaucoma-associated gene, e.g., MYOC, TBK1, or CDKN2B-AS1. For purposes of this paragraph, the gene nomenclature is also used to designate the polypeptide.

### 3.2 Alternative Methods to Genetically Modify Neuronal Cells

In some embodiments, genetic modification to introduce a transgene encoding a dominant negative polypeptide into neuronal cells is performed using a transposase-based system for gene integration, e.g., a CRISPR/Cas-mediated gene integration, TALENS or Zinc-finger. For example, CRISPR/Cas-mediated gene integration may be employed to introduce a dominant negative polypeptide into neuronal cells in vivo or to neuronal cells ex vivo, which may then be administered to a patient. In some embodiments, a gene modification system, e.g., a CRISPR/Cas, TALENS or Zinc-finger nuclease system is employed to introduce a dominant negative mutation as described herein into the endogenous gene. Such systems may be delivered to neural cells using any system, including viral vector systems.

### 3.3 Neural Cells Genetically Modified Express a Dominant Negative Mutation

Any neural cell in which DLK participates in kinase signaling can be modified to express a dominant negative polypeptide as described in the present disclosure. A “neural cell” as used herein includes any type of cell relating to neurons, as well as non-neural cells, such as glia, that play a role in neural pathways. In some embodiments, the neural cells are ophthalmic neurons. In some embodiments, the neural cells are retinal pigment epithelial (RPE) cells, photoreceptor cells, retinal ganglia, neural glia cells, including Muller cells, other inner retinal neurons such as bipolar cells, and amacrine cells, corneal endothelial cells and the like. In some embodiment, the neural cells are ophthalmic neurons, retinal pigment epithelial (RPE) cells, photoreceptor cells, or retinal ganglia. In some embodiments, the neural cells are otologic neurons, including inner and outer hair cells. In some embodiments, the neural cells are spiral ganglion neurons. In some embodiments, the neural cells are trigeminal neuron or facial nerve neurons. In some embodiments, the neural cells are peripheral nervous system (PNS) or central nervous system (CNS) cells, such as CNS neurons.

### 4. Neuronal Protection/Inhibition of Neuronal Cell Death

The present disclosure additionally provides methods of inhibiting neuronal cell death by genetically modifying a population of neurons to express a dnDLK polypeptide or dnDLK/LZK polypeptide as described herein. Although in some embodiments, the polypeptide may be administered as a protein, in typical embodiments, a nucleic acid encoding a dnDLK polypeptide or dnDLK/LZK polypeptide is introduced into a population of neuronal cells. The following section focuses on administration of nucleic acids encoding the polypeptides. One of skill understand that dnDLK polypeptide or dnDLK/LZK polypeptides may also be employed in certain therapeutic applications, e.g., that do not require sustained presence of the polypeptides. Pharmaceutical formulations for polypeptide can readily be prepared based on known parameters for delivery of polypeptides to a subject.

A composition may be administered to any subject, including non-human subject such as a mouse, rat, cat, dog, sheep, rabbit, horse, cow, goat, pig, guinea pig, hamster, an avian, or a non-human primate (e.g., a macaque). In typical embodiments, the subject is a human.

### 4.1 Pharmaceutical Compositions and Administration

In one aspect, the disclosure provide pharmaceutical compositions comprising a nucleic acid encoding a dominant negative polypeptide, for inhibiting neuronal cell death. The nucleic acid is administered in a therapeutically effective amount using a dosing regimen suitable for treatment of a neuronal degeneration disease. A “therapeutically effective amount” refers to an amount that is effective, at dosages and for periods of time necessary, to achieve a desired result, including a reduction, delay, amelioration or any improvement in one or more symptoms of a neural disease, such as a neuronal degeneration disease. In some embodiments, the nucleic acid encoding a dominant negative polypeptide is administered prophylactically to prevent one or more systems of a neuronal disease. A therapeutically effective amount of such a composition may vary according to factors such as the disease state, age, sex, and weight of the individual, or the ability of the gene therapy vector to elicit a desired response in the individual. Dosage regimens may be adjusted to provide the optimum response. A therapeutically effective amount is also one in which any toxic or detrimental effects of the viral vector are outweighed by the therapeutically beneficial effects.

The composition can be formulated for use in a variety of drug delivery systems. One or more physiologically acceptable excipients or carriers can also be included in the compositions for proper formulation. For example, the nucleic acid encoding the dominant negative polypeptide may be formulated in a solution suitable for administration to the patient, such as a sterile isotonic solution for injection. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

In some embodiments, delivery vehicles such as liposomes, nanocapsules, nanoparticles, microspheres, lipid particles, vesicles, and the like, may be used to facilitate administration of the pharmaceutical compositions. For example, in some embodiments, a viral vector comprising a transgene encoding a dominant negative polypeptide described herein can be formulated for delivery encapsulated in a lipid particle, a liposome, a vesicle, a nanosphere, or a nanoparticle or the like.

In typical embodiments, a viral vector encoding a dominant negative polypeptide as described herein is administered to a subject. Dosage values may vary with the severity of the condition. It is to be further understood that for any particular subject, specific dosage regimens can be adjusted over time according to the individual need and the professional judgment of the person administering or supervising the administration of the compositions, and that dosage ranges set forth herein are for illustrative purposes only and are not intended to limit the scope or practice of the claimed composition.

In some embodiments, the viral vector administered to a subject is an AAV particle preparation. Dosage regimens may be adjusted to provide the optimum therapeutic response. For example, a single bolus may be administered, several divided doses may be administered over time or the dose may be proportionally reduced or increased as indicated by the exigencies of the therapeutic situation. In some embodiments, e.g., administration to a human, an AAV dosage may be from 10⁹ to 10¹³ AAV particles per injection, e.g., intraocular injection. In some embodiments 10⁹ to 10¹⁰, 10¹⁰ to 10¹¹, 10¹¹ to 10¹², or 10¹² to 10¹³ viral particles may be administered per injection. As used herein for dosages, the number of AAV particles is equivalent to the number of viral genomes.

Multiple treatments, including in combination with other gene therapy vectors, may be administered to any subject, including over the lifetime of the subject.

The nucleic acid encoding the dominant negative polypeptide may be introduced using any route of administration. For example, a gene therapy vector may be administered systemically, e.g., by intravenous injection or infusion; or locally, e.g., by retinal injection, or ocular administration or infusion. The invention is not limited to a particular site or method of administration.

### 4.2 Neural Disorders

A nucleic acid encoding a dominant negative polypeptide as described herein, such as a gene therapy vector to administer a transgene encoding the polypeptide, may be used to treat neuronal disorder, including, but not limited to the following: glaucoma (including open-angle and narrow/closed-angle glaucoma, primary and secondary glaucoma, normal tension and high-IOP glaucoma), age-related macular degeneration (AMD) including dry (non-exudative) and wet (exudative, neovascular) AMD, choroidal neovascularization (CNV), choroidal neovascular membranes (CNVM), cystoid macular oedema (CME), epiretinal membranes (ERM) and macular perforations, myopia-associated choroidal neovascularization, angioid and vascular streaks, retinal detachment, diabetic retinopathy, diabetic macularoedema (DME), atrophic and hypertrophic lesions in the retinal pigment epithelium, retinal vein occlusion, choroidal retinal vein occlusion, macular oedema, macular oedema associated with renal vein occlusion, retinitis pigmentosa and other inherited retinal degenerations (e.g. Stargardt disease), retinopathy of prematurity, other optic neuropathies including toxic optic neuropathy (e.g. methanol, ethambutol), nonarteritic ischemic optic neuropathy, arteritic ischemic optic neuropathy/giant cell arteritis, traumatic optic neuropathy (including traumatic brain injury), idiopathic intracranial hypertension/pseudotumor cerebri, inflammatory optic neuropathies (e.g. optic neuritis), compressive optic neuropathies (e.g. pituitary adenoma), infiltrative optic neuropathies (e.g. sarcoidosis, lymphoma), autoimmune optic neuropathies, lipid storage diseases (e.g. Tay-Sachs), nutritional optic neuropathies, Leber's hereditary optic neuropathy, dominant optic atrophy, Friedrich's ataxia, radiation-induced optic neuropathy, iatrogenic optic neuropathies, space flight-associated neuro-ocular syndrome (SANS), inflammation disorders of the eye, for example uveitis, scleritis, cataract, refraction anomalies, for example myopia, hyperopia, astigmatism or keratoconus, neurotrophic keratopathy, corneal denneratvation and promoting corneal reinnervation and diabetic keratopathy.

Neurodegenerative non-ophthalmological disorders may also be treated using a nucleic acid encoding a dominant negative polypeptide as described here. Such disorders, include, but not limited to, the following: Amyotrophic lateral sclerosis (ALS), Alzheimer's disease, Parkinson's disease, traumatic brain injury, Parkinson's-plus disease, Huntington's disease, peripheral neuropathies, ischemia, stroke, intracranial haemorrhage, cerebral haemorrhage, nerve damage caused by exposure to toxic compounds selected from the group consisting of heavy metals, industrial solvents, drugs and chemotherapeutic agents, injury to the nervous system caused by physical, mechanical or chemical trauma trigeminal neuralgia, glossopharyngeal neuralgia, Bell's Palsy, myasthenia gravis, muscular dystrophy, progressive muscular atrophy, primary lateral sclerosis (PLS), spinal muscular atrophy, inherited muscular atrophy, invertebrate disk syndromes, cervical spondylosis, plexus disorders, thoracic outlet destruction syndromes, porphyria, pseudobulbar palsy, progressive bulbar palsy, multiple system atrophy, progressive supranuclear palsy, corticobasal degeneration, dementia with Lewy bodies, frontotemporal dementia, demyelinating diseases, Guillain-Barre syndrome, multiple sclerosis, Charcot-Marie-Tooth disease, prion disease, Creutzfeldt-Jakob disease, Gerstmann-Straussler-Scheinker syndrome (GSS), fatal familial insomnia (FFI), bovine spongiform encephalopathy, Pick's disease, epilepsy, AIDS demential complex. In some embodiments, a dominant negative polypeptide as described herein is employed to prevent herpesvirus reactivation. In some embodiments, a dominant negative polypeptide as described herein is employed to prevent aberrant neural cell regeneration. In some embodiments, the disorder is chemotherapy-induced peripheral neuropathy (CIPN), e.g., nerve damage caused by exposure to chemotherapeutic agents. In some embodiments, the disease is a hearing loss disorder, for example, age-related hearing loss, chemotherapy-induced hearing loss, hereditary hearing loss, aminoglycoside-induced hearing loss, trauma-induced hearing loss, and noise-induced hearing loss.

In some embodiments, a nucleic acid encoding the polypeptide is administered to a subject to treat or prevent glaucoma, age-related macular degeneration, choroidal neovascularization (CNV), myopia-associated choroidal neovascularization, diabetic retinopathy, macular oedema, and retinal vein occlusion.

In some embodiments, a nucleic acid encoding the polypeptide is administered to a subject to treat a retinal disease, such as an inherited retinal disease, e.g., retinitis pigmentosa. In some embodiments, the retinal disease involves rhodopsin mistrafficking and/or misfolding.

In some embodiments, a nucleic acid encoding a dominant negative polypeptide described herein can be used to treat and/or prevent optic neuropathies, including glaucoma, inherited retinal degenerations, non-exudative AMD/geographic atrophy, retinal vascular diseases that produce ischemia (diabetes, vein occlusion), retinal detachments and edema-producing diseases (including exudative AMD).

Another aspect of the disclosure are cell transplantation-based regenerative approaches for the treatment of ocular and other forms of neurodegeneration. These include photoreceptor and/or RPE transplantation for treatment of macular degeneration and forms of photoreceptor degeneration, and RGC transplantation for the treatment of glaucoma and other forms of optic nerve disease. Accordingly, in some embodiments, a nucleic acid encoding the polypeptide can be introduced into cells for transplant, either before or after transplants of neuronal cell for ocular degenerative diseases and other forms of neurodegeneration. In some embodiments, the nucleic acid encoding the polypeptide can be introduced into precursor cells such as stem cells.

The aforementioned well-characterized diseases in humans can also occur with comparable etiology in other mammals and can likewise be treated therein with the compounds of the present disclosure.

### P1 EMBODIMENTS

Embodiment P1-1. A nucleic acid encoding a dominant negative Dual Leucine Zipper Kinase (dnDLK) polypeptide comprising an amino acid segment with a sequence with at least 95% identity to region 1-520 of SEQ ID NO:1, wherein the polypeptide comprises at least one mutation, as determined with reference to SEQ ID NO:1, wherein the mutation is selected from a substitution at position 43, a substitution at position 302, and a substitution at position 516.

Embodiment P1-2. The nucleic acid of Embodiment P1-1, wherein the dnDLK polypeptide comprises a substitution at position 302, wherein the substitution is any amino acid other than threonine.

Embodiment P1-3. The nucleic acid of Embodiment P1-2, wherein the substitution at position 302 is S302A.

Embodiment P1-4. The nucleic acid of any one of Embodiment P1-1, -2, or -3, wherein the dnDLK polypeptide comprises a substitution at position 43.

Embodiment P1-5. The nucleic acid of Embodiment P1-4, wherein the substitution at position 43 is E or D.

Embodiment P1-6. The nucleic acid of Embodiment P1-5, wherein the substitution at position 43 is E.

Embodiment P1-7. The nucleic acid of Embodiment P1-1, wherein the dnDLK polypeptide comprises a substitutions T43E and S302A.

Embodiment P1-8. The nucleic acid of any one of Embodiments P1-1 to P1-7, wherein the dnDLK polypeptide comprises a substitution at position 185.

Embodiment P1-9. The nucleic acid of Embodiment P1-8, wherein the substitution at position 185 is K185A.

Embodiment P1-10. The nucleic acid of any one of Embodiments P1-1 to P1-9, wherein the dnDLK polypeptide comprises a substitution at position 516.

Embodiment P1-11. The nucleic acid of Embodiment P1-10, wherein the substitution at position 516 is G516V.

Embodiment P1-12. The nucleic acid of any one of Embodiment P1-1 to P1-11, wherein the dnDLK polypeptide comprising a substitution at position 424 or 426.

Embodiment P1-13. The nucleic acid of Embodiment P1-12, wherein the dnDLK polypeptide further comprising a substitution at position 431, 438, 440, 445, 447, 486, 491, or 493.

Embodiment P1-14. The nucleic acid of Embodiment P1-1, comprising the amino acid sequence of SEQ ID NO: 6 or SEQ ID NO:7.

Embodiment P1-15. An isolated nucleic acid encoding a dominant negative dual leucine zipper (dnDLK) polypeptide comprising an amino acid sequence having at least 95% identity to region 158-520 of SEQ ID NO:1, wherein the polypeptide comprises at least one mutation, as determined with reference to SEQ ID NO:1, wherein the mutation is selected from a substitution at position 302 and a substitution at position 516.

Embodiment P1-16. The nucleic acid of Embodiment P1-15, wherein the dnDLK polypeptide comprises a substitution at position S302, wherein the substitution is any amino acid other than threonine.

Embodiment P1-17. The nucleic acid of Embodiment P1-16, wherein the substitution is S302A.

Embodiment P1-18. The nucleic acid of Embodiment P1-15, -16, or -17, wherein the dnDLK polypeptide comprises a substitution at position 516.

Embodiment P1-19. The nucleic acid of Embodiment P1-18, wherein the substitution at position 516 is G516V.

Embodiment P1-20. The nucleic acid of any one of Embodiment P1-15 to P1-19, wherein the dnDLK polypeptide comprises a substitution at position 185.

Embodiment P1-21. The nucleic acid of Embodiment P1-20, wherein the substitution at position 185 is K185A.

Embodiment P1-22. The nucleic acid of any one of Embodiment P1-15 tp P1-21, wherein the dnDLK polypeptide comprises a substitution at position 424 and/or 426.

Embodiment P1-23. The nucleic acid of Embodiment P1-22, wherein the dnDLK polypeptide further comprises a substitution at position 431, 438, 440, 445, 447, 486, 491, or 493.

Embodiment P1-24. An isolated nucleic acid encoding a leucine zipper polypeptide that inhibits homodimerization and DLK heterodimerization with (LZK), wherein the polypeptide comprises an amino acid sequence having at least 95% identity to SEQ ID NO:8.

Embodiment P1-25. The nucleic acid of Embodiment P1-24, wherein the polypeptide is fewer than 150 amino acids in length.

Embodiment P1-26. A vector comprising a nucleic acid of any one of Embodiment P1-1 to P1-25.

Embodiment P1-27. The vector of Embodiment P1-26, wherein the vector is a viral vector.

Embodiment P1-28. The vector of Embodiment P1-27, wherein the viral vector is an adeno-associated virus (AAV) vector.

Embodiment P1-29. The vector of Embodiment P1-28, wherein the AAV vector is an AAV1, AAV2, AAV3, AAV4, AAV5, AAV6, AAV7, AAV8, AAV9, AAV10, AAV11-derived or pseudotyped AAV-derived vector.

Embodiment P1-30. The vector of Embodiment P1-29, wherein the vector is AAV2.7m8.

Embodiment P1-31. The vector of any one Embodiments P1-26 tp P1-31, further comprising a Woodchuck Hepatitis Virus Posttranscriptional Regulatory Element (WPRE).

Embodiment P1-32. A host cell comprising a nucleic acid of any one of Embodiment P1-1 to P1-25 or a vector of any one of Embodiments P1-26 to P1-31.

Embodiment P1-33. The host cell of Embodiment P1-32, wherein the host cell is a neuron.

Embodiment P1-34. The host cell of Embodiment P1-33, wherein the neuron is a retinal ganglion.

Embodiment P1-35. The host cell of Embodiment P1-33 or P1-34, wherein the host cell is a mammalian cell.

Embodiment P1-36. The host cell of Embodiment P1-35, wherein the host cell is a human cell.

Embodiment P1-37. A method of inhibiting neuronal cell death, the method comprising introducing a nucleic acid of any one of Embodiment P1-1 to P1-25 or a vector of any one of Embodiment P1-26 to P1-31 into a neural cell.

Embodiment P1-38. The method of Embodiment P1-37, wherein the neural cell is ex vivo.

Embodiment P1-39. The method of Embodiment P1-37, wherein the neural cell is in vivo.

Embodiment P1-40. The method of any one of Embodiment P1-37 to P1-39, wherein the neural cell is an ophthalmic neuron.

Embodiment P1-41. The method of Embodiment P1-40, wherein the ophthalmic neuron is a retinal ganglion.

Embodiment P1-42. The method of any one of Embodiment P1-37 to P1-39, wherein the neural cell is a photoreceptor cell.

Embodiment P1-43. The method of Embodiment P1-37 to P1-42, wherein the neural cell is mammalian.

Embodiment P1-44. The method of Embodiment P1-43, wherein the neuron is a human neural cell.

Embodiment P1-45. A method of treating or preventing neural cell death in a subject in need thereof, the method comprising administering a nucleic acid of any one of Embodiment P1-1 to P1-25 or a vector of any one of Embodiment P1-26 to P1-31 to the subject.

Embodiment P1-46. The method of Embodiment P1-45, wherein the subject has glaucoma, age-related macular degeneration, choroidal neovascularization (CNV), myopia-associated CNV, diabetic retinopathy, macular oedema, and retinal vein occlusion.

Embodiment P1-47. The method of Embodiment P1-45, wherein the subject has an inherited retinal disease.

Embodiment P1-48. The method of Embodiment P1-47, wherein the inherited retinal disease is retinitis pigmentosa.

Embodiment P1-49. A polypeptide encoded by a nucleic acid of any one of Embodiments P1-1 to P1-25.

### EMBODIMENTS

Embodiment 1. A nucleic acid encoding a dominant negative Dual Leucine Zipper Kinase (dnDLK) polypeptide comprising an amino acid segment with a sequence having at least 80% identity to region 1-520 of SEQ ID NO:1, wherein the dnDLK polypeptide comprises at least one mutation, as determined with reference to SEQ ID NO:1, wherein the mutation is a substitution at position 302, wherein the substitution is any amino acid other than threonine.

Embodiment 2. The nucleic acid of Embodiment 1, wherein the substitution at position 302 is S302A.

Embodiment 3. The nucleic acid of Embodiment 1 or 2, wherein the dnDLK polypeptide is fewer than 550 amino acids in length.

Embodiment 4. The nucleic acid of any one of Embodiments 1-3, wherein the dnDLK polypeptide comprises a substitution at position 43.

Embodiment 5. The nucleic acid of Embodiment 3, wherein the substitution at position 43 is E or D.

Embodiment 6. The nucleic acid of Embodiment 5, wherein the substitution at position 43 is E.

Embodiment 7. The nucleic acid of any one of Embodiments 1 to 6 wherein the dnDLK polypeptide comprises a substitution at position 185.

Embodiment 8. The nucleic acid of Embodiment 7, wherein the substitution at position 185 is K185A.

Embodiment 9. The nucleic acid of any one of Embodiments 1 to 8, wherein the dnDLK polypeptide comprising a substitution at position 424 or 426.

Embodiment 10. The nucleic acid of Embodiment 9, wherein the dnDLK polypeptide further comprising a substitution at position 431, 438, 440, 445, 447, 486, 491, or 493.

Embodiment 11. The nucleic acid of Embodiment 1, wherein the dnDLK polypeptide comprises the amino acid sequence of SEQ ID NO: 6 or SEQ ID NO:7.

Embodiment 12. The nucleic acid of any one of Embodiments 1-11, comprising a nucleic acid sequence having at least 95% identity to SEQ ID NO:11.

Embodiment 13. An isolated nucleic acid encoding a leucine zipper polypeptide that inhibits homodimerization and DLK heterodimerization with (LZK), wherein the polypeptide comprises an amino acid sequence having at least 80% identity to SEQ ID NO:8; or comprises SEQ ID NO:8.

Embodiment 14. The nucleic acid of Embodiment 13, wherein the polypeptide is fewer than 150 amino acids in length.

Embodiment 15. An isolated nucleic acid encoding a dominant negative dual leucine zipper (dnDLK) polypeptide comprising an amino acid sequence having at least 90% identity to region 158-520 of SEQ ID NO:1, wherein the polypeptide comprises at least one mutation, as determined with reference to SEQ ID NO:1, wherein the at least one mutation is at position 302.

Embodiment 16. The nucleic acid of Embodiment 15, wherein the dnDLK polypeptide comprises a substitution at position S302, wherein the substitution is any amino acid other than threonine.

Embodiment 17. The nucleic acid of Embodiment 16, wherein the substitution is S302A.

Embodiment 18. The nucleic acid of any one of Embodiments 15-17, wherein the dnDLK polypeptide comprises a substitution at position 185.

Embodiment 19. The nucleic acid of Embodiment 18, wherein the substitution at position 185 is K185A.

Embodiment 20. The nucleic acid of any one of Embodiments 15-19, wherein the dnDLK polypeptide comprises a substitution at position 424 and/or 426.

Embodiment 21. The nucleic acid of Embodiment 20, wherein the dnDLK polypeptide further comprises a substitution at position 431, 438, 440, 445, 447, 486, 491, or 493.

Embodiment 22. A vector comprising a nucleic acid of any one of Embodiments 1 to 14.

Embodiment 23. The vector of Embodiment 22, wherein the vector is a viral vector.

Embodiment 24. The vector of Embodiment 23, wherein the viral vector is an adeno-associated virus (AAV) vector.

Embodiment 25. The vector of Embodiment 24, wherein the AAV vector is an AAV1, AAV2, AAV3, AAV4, AAV5, AAV6, AAV7, AAV8, AAV9, AAV10, AAV11-derived or pseudotyped AAV-derived vector.

Embodiment 26. The vector of Embodiment 25, wherein the vector is AAV2.7m8.

Embodiment 27. The vector of any one Embodiments 22 to 26, further comprising a Woodchuck Hepatitis Virus Posttranscriptional Regulatory Element (WPRE).

Embodiment 28. A host cell comprising a nucleic acid of any one of Embodiments 1 to 21 or a vector of any one of Embodiments 22 to 27.

Embodiment 29. The host cell of Embodiment 28, wherein the host cell is a neuron.

Embodiment 30. The host cell of Embodiment 29, wherein the neuron is a retinal ganglion.

Embodiment 31. The host cell of Embodiment 29 or 30, wherein the host cell is a mammalian cell.

Embodiment 32. The host cell of Embodiment 31, wherein the host cell is a human cell.

Embodiment 33. A method of inhibiting neuronal cell death, the method comprising introducing a nucleic acid of any one of Embodiments 1 to 21 or a vector of any one of Embodiments 22 to 27 into a neural cell.

Embodiment 34. The method of Embodiment 33, wherein the neural cell is ex vivo.

Embodiment 35. The method of Embodiment 33, wherein the neural cell is in vivo.

Embodiment 36. The method of any one of Embodiments 33 to 35, wherein the neural cell is an ophthalmic neuron.

Embodiment 37. The method of Embodiment 36, wherein the ophthalmic neuron is a retinal ganglion.

Embodiment 38. The method of any one of Embodiments 33 to 35, wherein the neural cell is a photoreceptor cell.

Embodiment 39. The method of any one of Embodiments 33 to 38, wherein the neural cell is mammalian.

Embodiment 40. The method of Embodiment 39, wherein the neuron is a human neural cell.

Embodiment 41. A method of treating or preventing neural cell death in a subject in need thereof, the method comprising administering a nucleic acid of any one of Embodiments 1 to 21 or a vector of any one of Embodiments 22 to 27 to the subject.

Embodiment 42. The method of Embodiment 41, wherein the subject has glaucoma, age-related macular degeneration, choroidal neovascularization (CNV), myopia-associated CNV, diabetic retinopathy, macular oedema, and retinal vein occlusion.

Embodiment 43. The method of Embodiment 41, wherein the subject has an inherited retinal disease.

Embodiment 44. The method of Embodiment 43, wherein the inherited retinal disease is retinitis pigmentosa.

Embodiment 45. A polypeptide encoded by a nucleic acid of any one of Embodiments 1 to 21.

### 5. Assays, Methods and Experimental Results

### 5.1 Methodology

### 5.1.1 Lentivirus Production

Lentivirus expressing DN DLK and LZK were made by subcloning synthetic DNA fragments into pLenti-EF1-mScarlet backbone using Gibson Assembly (Kpn21-digested). The result was DLK fused to mScarlet with a small linker peptide. For the leucine zippers, a P2A sequence to induce ribosomal skipping during translation was inserted between mScarlet and the fragment. 293-HEK cells were transfected with the various lentivirus constructs using Lipofectamine 2000 (Thermo Scientific #11668019). 1M Sodium Butyrate (Sigma Aldrich #B5887) was added 24 hours post transfection. Viral supernatant was collected 48 hours post transfection and concentrated with Lenti-X Concentrator (Takara).

### 5.1.2 Primary RGCs

Retinas were isolated from postnatal day 0-3 mice and dissociated with papain. Microglia were immunodepleted with CELLection Dynabeads (Invitrogen) conjugated to anti-CD11b (BD Pharmingen, 554859). The suspension of retinal cells was immunopanned on plates pre-conjugated with anti-Thy1.2 antibodies (BioRad, MCA02R) and goat anti-mouse IgM (Jackson Immunoresearch, 115-001-020) at room temperature (RT). After washing, retinal ganglion cells (RGCs) were released from the plate with trypsin (Sigma T9201), counted, and seeded at a density of 5,000-10,000 per well in 96-well plates (Nunclon plates and Poly-D-lysine coated). Growth media was composed of Neurobasal (Life Technologies) supplemented with NS21, Sato, L-glutamine, penicillin/streptomycin, N-acetyl-cysteine, insulin, sodium pyruvate, triiodothyronine (T3), forskolin (Chen et al., 2008) and 1 μM of GNE 3511 (Genentech) to prevent RGC death. Transduction of lentivirus DN DLK and LZK was performed at the time of isolation.

### 5.1.3 Viability Assay

On day 3-5 post transduction, GNE 3511 (a DLK/LZK inhibitor) was withdrawn from the primary RGCs to initiate cell death. Cells were assayed for survival 3 days post GNE withdrawal [in relative light units (RLU)] by adding a 50% volume of CellTiter-Glo (CTG) (Promega G8462). Luminescence was measured with a plate reader (Molecular Devices). This allowed for a calculation of the number of viable cells. “Potency” of the lentivirus constructs was evaluated by determining the lowest titer of lentivirus (represented as the level of viral genomes (vg)) that produced a survival effect measured by CTG. “Efficacy” of constructs was evaluated by comparing the survival of different constructs at the same lentivirus concentration.

### 5.1.4 Quantitative Reverse Transcriptase PCR to Quantify Lentiviral Genome Copy Number

293-HEK cells were transduced with lentivirus expressing DN DLK and LZK as described in section 5.1.1. Cells were harvested and DNA was extracted using a QuickExtract RNA extraction kit (Lucigen #QE09050). PCR analysis was conducted using a CFX Connect Real-Time PCR Detection System (Bio-Rad 1855200). qPCR was performed with the SsoAdvanced Universal SYBR Green Supermix (Bio-Rad 1725270) using forward (CCTTTCCGGGACTTTCGCTTT) and reverse (GCAGAATCCAGGTGGCAACA) primers to detect lentivirus mRNA levels (i.e., genome copy number). Sox11 was amplified as an internal control (Sox11, Forward primer: CACCGATGAACGGGATCTTCTCGC, Reverse primer: AAACGCGAGAAGATCCCGTTCATC).

### 5.1.5 Random Mutagenesis Screening

An approximately 1 kb section of DLK (nucleotides 772-1830) was amplified from full length DLK cDNA. The amplicon was randomly mutagenized using error-prone PCR using GeneMorph II Error-Prone PCR Kit (Agilent) keeping an approximate mutation frequency of 1 mutation/kb. The mutagenized insert and backbone were then digested with Aarl and BstXI and ligated using Quick Ligase (NEB). Transduction of lentivirus DN DLK library into primary RGCs was performed at the time of isolation at a multiplicity of infection of 0.3. Selective pressure was induced by withdrawing GNE 3511 on day 3-5 post transduction. Surviving cells 5-7 days post GNE 3511 withdrawal were collected and analyzed via next generation sequencing.

### 5.2 Generation of Neuroprotective DLK Variants

We prepared a series of constructs to evaluate the effects of mutation of wild type human DLK (SEQ ID NO:1) at particular positions, and to test the effects of truncation of DLK at the C-terminus.

5.2.1 S302 dnDLK

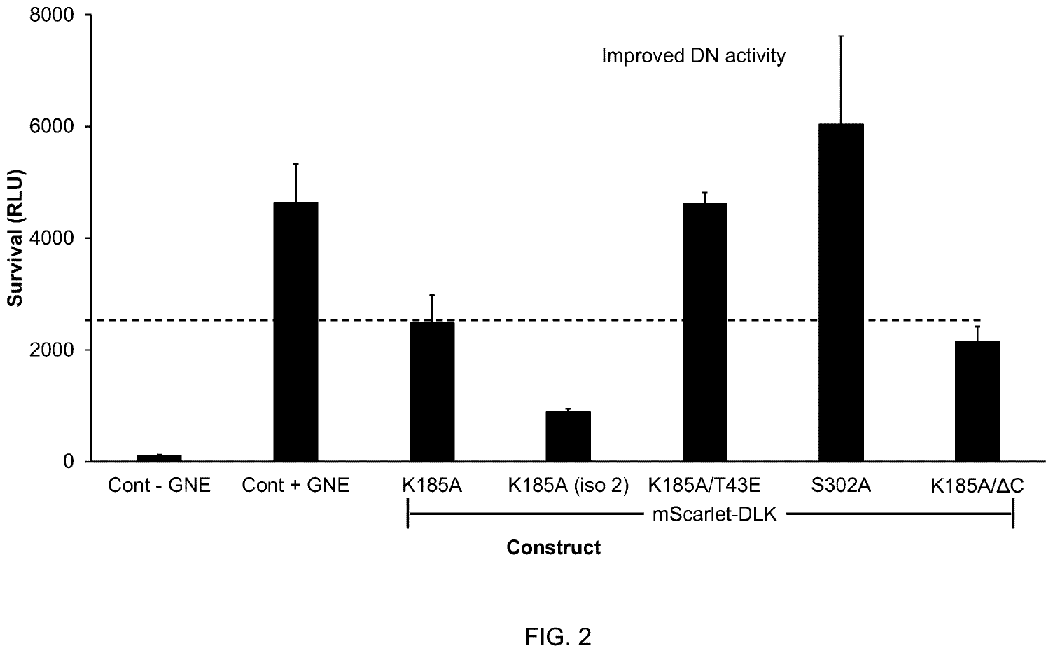

We generated a DLK mutant having a substitution at position 302, a DLK phosphorylation site. Alanine was substituted for S at position 302 (S302A) and the effects on survival of mouse retinal ganglion neurons were evaluated as described above. In some experiments, protective activity was compared to dnDLK K185A. The K185A mutation blocks DLK autophorphorylation. We found that a S302A variant of DNK was neuroprotective. Indeed, S302 DLK had greater neuroprotective activity than the DLK K185A variant. See FIG. 2 (K185A vs S302A). Without intending to be bound by a particular mechanism, it is believed that the S302A mutation blocks DLK activation by phosphokinase A (PKA).

FIG. 2 also provides data showing that a K185A mutation introduced into the shorter human DLK isoform SEQ ID NO:13 (construct K185A (iso 2) in FIG. 2) was also neuroprotective, although at a lower level compared to neuroprotection obtained with the other mutant constructs assessed in this experiment.

### 5.2.2 C-Terminal Truncation (ΔC-dnDLK)

We found that removal of the C-terminal amino acids 521-892 of K185A DLK did not significantly reduce the protective activity of K185A DLK. See FIG. 2 (K185A vs K185A/ΔC). We concluded we could reduce the size of DLK by deleting the C-terminus whilst retaining dnDLK activity. The smaller size of ΔC-dnDLK means that a much shorter transgene can be used to deliver active dnDLKs to cells.

5.2.3 S302/ΔC dnDLK

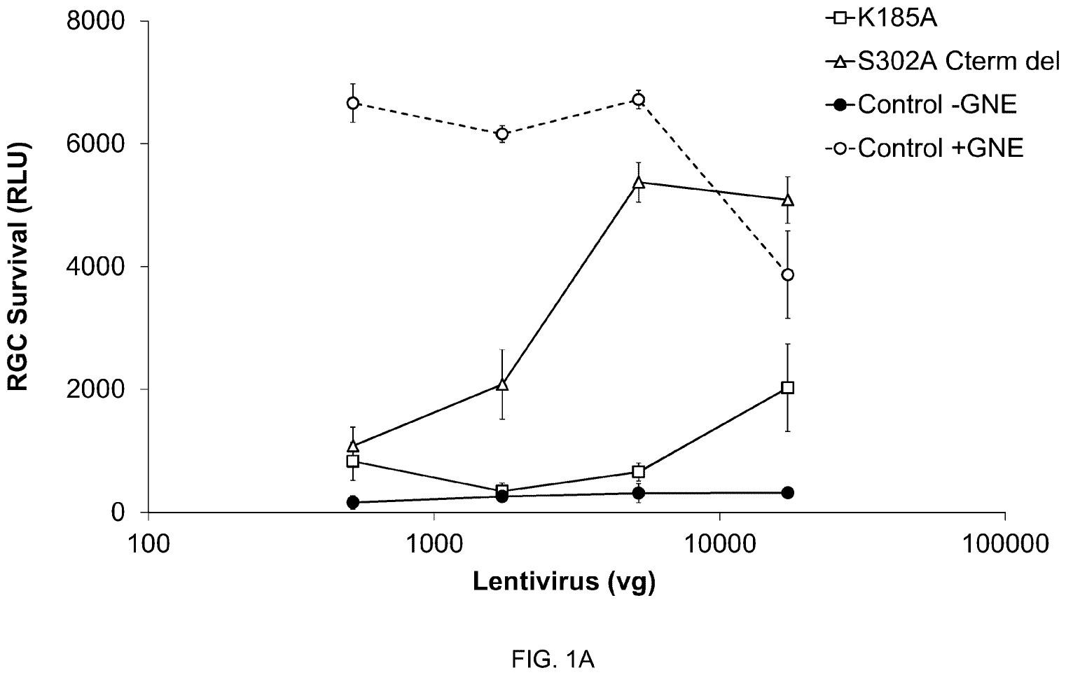

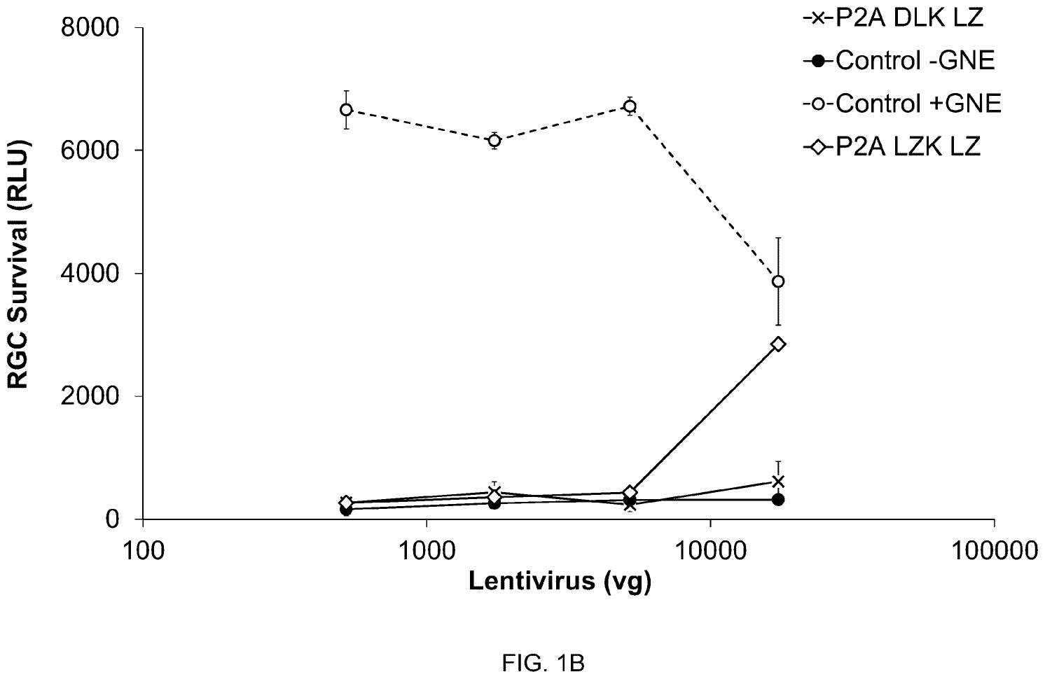

We prepared a variant in which the S302A substitution was combined with the C-terminal deletion of residues 521-892. As shown in FIG. 1A, S302/ΔC dnDLK was more protective than the K185A variant. Potency of the transgene increased 10-fold while the max efficacy increased 2.5-fold.

### 5.2.4 LZK LZ is Neuroprotective

DLK is known to homodimerize through the DLK leucine zipper. It has also been shown that the DLK leucine zipper itself can function as a dnDLK (Nihalani et al., J. Biol. Chem. 275: 7273-7279, 2000), presumably by its ability to bind DLK and prevent homodimerization.

We prepared a construct to express the LZK leucine zipper domain (SEQ ID NO:8.) Surprisingly, the LZK leucine zipper domain (construct designated as P2A LZK LZ in FIG. 1B) had neuroprotective activity. The construct increased potency 10-fold and efficacy around 8.5-fold (see FIG. 1B). A construct that expressed the DLK leucine zipper domain (designated as P2A DLK LZ in FIG. 2) did not exhibit protective activity. We also generated DLK LZ and LZK LZ P2A-fused constructs, which did not have an effect on RGC survival (data not shown). Similarly, co-transfection of constructs encoding DLK LZ and LZK LZ did not provide improved survival (data not shown). Without intending to be bound by a particular mechanism, we believe the LZK LZ polypeptide inhibits DLK-LZK heterodimerization.

5.2.5 T43E dnDLK

In this example, we made a series of phosphomimetic mutants (T9E, S11E, T43E, S272E and S533E). Of these, the T43E variant was neuroprotective See FIG. 2 (K185A vs K185A/T43E).

Huntwork-Rodriguez (J. Cell Biol 202:747-763, 2013) demonstrated that after neuronal insult, specific sites through the length of DLK underwent phosphorylation by c-Jun N-terminal kinases. These phosphorylation events resulted in increased DLK abundance via reduction of DLK ubiquitination. We hypothesized that phosphomimetic mutations could be introduced at these sites to create more stable dominant negative DLK polypeptides. The sites T9, S11, T43, S272, and S533 were mutagenized as indicated above. Phosphomimetic mutations were introduced into the K185A mutant background.

Lentivirus encoding the various phosphomimetic DN DLK mutants were generated as described in section 5.1.1 and transduced into RGCs in equal titer. DLK/LZK inhibitor GNE 3511 was present in the media to prevent cell death. On day 3-5 post transduction, GNE 3511 was withdrawn from the primary RGCs to initiate cell death. Cells were assayed for survival 3 days post GNE withdrawal. Luminescence (relative light unitys (RLU) was measured to determine the number of viable cells.

In this analysis, only T43E K185A showed enhanced neuroprotection relative to K185A. The neuroprotective activity of all of the constructs is summarized below:
T9E S11E K185A—No significant difference compared to K185A mutation alone
S533E K185A—No significant difference compared to K185A mutation alone
T9E S11E S272E S533E K185A—No significant difference compared to K185A mutation alone
T43E S272E S533E K185A—No improved survival compared to negative control (GNE withdrawal)
T43E K185A—About 3-fold improvement in survival compared to K185A alone
   5.2.6 G516 dnDLK

As an alternate approach to discover dnDLK mutations, we randomly mutagenized human DLK, transduced RGCs and recovered the sequences remaining in the surviving cells (i.e. directed evolution).

We generated a lentivirus library in which DLK was randomly mutagenized. RGCs were infected at a multiplicity of infection (MOI) of 1 such that on average each cell received one virus. LK/LZK inhibitor GNE 3511 was initially present in the media to prevent cell death. We then applied selective pressure by withdrawing GN 3511, so that only the surviving cells would contain an active DLK dominant negative. Prior to application of selective pressure, Next Gen sequencing showed a baseline distribution of mutations. After selective pressure, Next Gen sequencing of surviving cells determined that G516V was the most enriched over the baseline distribution. This screen thus identified a new mutant, G516V, which also creates a dnDLK.

5.3 Administration of dnDLK In Vivo

### 5.3.1 Rat Model of Glaucoma

The effects of administration of an AAV encoding a dnDLK variant (SEQ ID NO:6) in which the S302A substitution was combined with the C-terminal deletion of residues 521-892 was evaluated in a rat model of glaucoma.

Animals (8 per group) were anesthetized with isoflurane and intravitreally injected once with a dose of 5×10⁹ viral genomes AAV expressing dnDLK or expressing GFP as a control.

After 4 weeks, animals were anesthetized with ketamine/xylazine cocktail and eyes were locally anesthetized with proparacaine eye drops. Following anesthetization, a low temperature cautery pen (700-1000 F) was gently tapped on the eye around the circumference of the limbal plexus. Proper cauterization was noted by slight browning of the conjunctiva and blanching of the episcleral blood vessels. The eye was then coated with an antibiotic ointment to prevent infection and corneal drying. Six weeks following injury, animals were anesthetized and then perfused with 4% PFA and retinas were immunofluorescently labelled with RBPMS and SNCG.

To quantify RGC survival in the glaucoma model, a total of 16 images of RBPMS labelled RGCs per retina were taken at 10× magnification with equal representation of central and peripheral regions. The number of RGCs was quantified using an automated image analysis software for each picture and then averaged per retina.

To assess axonal degeneration SNCG labelled optic nerve heads were imaged at 10× magnification and montaged to assess axonal health. Montaged images were assessed by a panel of investigators for the level of heal scored from 1 to 5, with 5 being healthy axons and 1 being fully degenerated. Criteria for assessing health included thickness of axon bundles, uniformity or straightness of axon shape, and percentage of retina with axonal loss.

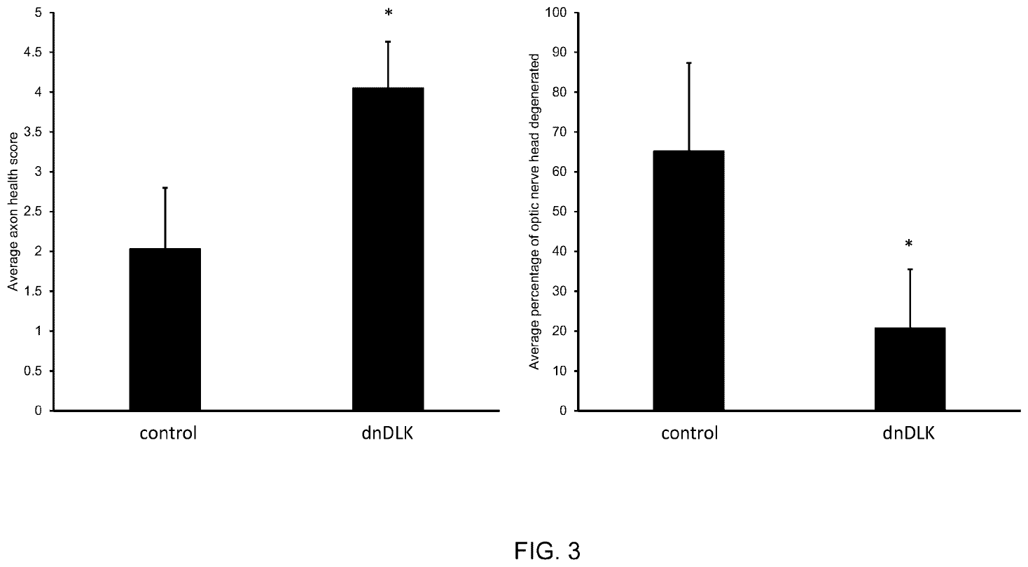

FIG. 3 shows the average axon health score of axons in animals receiving the dnDLK vs. control in the left panel and the average percentage of optic nerved head degeneration as assessed by evaluating axon degeneration in the right panel. Retinas from eyes that were injected with AAV expressing dnDLK were protected from glaucoma with an average score of 4 (out of 5) while retinas from eyes injected with AAV expressing GFP had degenerated axons with an average score of 2. Furthermore, the glaucoma injury caused an average of 65% axonal degeneration in GFP retinas while there was only 20% loss in axonal integrity in retinas with dnDLK. RGC survival following glaucoma was improved by dnDLK with an average of 8% loss in RGCs compared to retinas with dnDLK without injury. Retinas with GFP had an average 37.5% RGC loss with injury compared to GFP retinas without injury.

The results thus demonstrate that dnDLK protected RGCs from glaucoma injury in an in vivo rat model.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

TABLE OF ILLUSTRATIVE SEQUENCES

| SEQ ID | Protein/ | |
|---|---|---|
| NO | DNA | description |
| 1 | Protein | Complete human DLK isoform 2 sequence |
| 2 | Protein | Human DLK kinase domain |
| 3 | Protein | human DLK leucine zipper domain sequence |
| | | (as defined in Uniprot Q12852) |
| 4 | Protein | Leucine-zipper 1 sequence of human DLK(as |
| | | defined in Uniprot Q12852) |
| 5 | Protein | Leucine-zipper 2 sequence of human DLK (as |
| | | defined in Uniprot Q12852) |
| 6 | Protein | C-terminal deleted dnDLK with mutation at 302 |
| 7 | Protein | C-terminal deleted dnDLK with mutation at 302 |
| | | and 43 |
| 8 | Protein | human MAP3K13 (leucine zipper kinase) leucine |
| | | zipper domain amino acid sequence |
| 9 | DNA | DLK full-length cDNA sequence encoding |
| | | SEQ ID NO: 1 |
| 10 | DNA | dnDLK S302A ΔC-terminus WPRE polynucleotide |
| | | sequence |
| 11 | DNA | dnDLK T43E S302A ΔC-terminus WPRE |
| | | polynucleotide sequence |
| 12 | DNA | dominant negative LZK leucine zipper domain |
| | | polynucleotide sequence WPRE |
| 13 | Protein | Human DLK polypeptide sequence, isoform 1, |
| 14 | DNA | WPRE nucleic acid sequence |

SEQ ID NO: 1
Human DLK kinase domain polypeptide sequence, UniProt Q12852-2;
includes the amino acid sequence QCVLRDVVPLGGQGGGGPSPSPGGEPPPEPFANS in
place of the histidine at position 46 of UniProt Q12852-1; see SEQ ID NO: 13). The C-
terminal deleted sequence referred to in the EXAMPLES is underlined.
MACLHETRTP SPSFGGFVST LSEASMRKLD PDTSDCTPEK DLTPTQCVLR
DVVPLGGQGG GGPSPSPGGE PPPEPFANSV LQLHEQDAGG PGGAAGSPES
RASRVRADEV RLQCQSGSGF LEGLFGCLRP VWTMIGKAYS TEHKQQQEDL
WEVPFEEILD LQWVGSGAQG AVFLGRFHGE EVAVKKVRDL KETDIKHLRK
LKHPNIITFK GVCTQAPCYC ILMEFCAQGQ LYEVLRAGRP VTPSLLVDWS
MGIAGGMNYL HLHKIIHRDL KSPNMLITYD DVVKISDFGT SKELSDKSTK
MSFAGTVAWM APEVIRNEPV SEKVDIWSFG VVLWELLTGE IPYKDVDSSA
IIWGVGSNSL HLPVPSSCPD GFKILLRQCW NSKPRNRPSF RQILLHLDIA
SADVLSTPQE TYFKSQAEWR EEVKLHFEKI KSEGTCLHRL EEELVMRRRE
ELRHALDIRE HYERKLERAN NLYMELNALM LQLELKEREL LRREQALERR
CPGLLKPHPS RGLLHGNTME KLIKKRNVPQ KLSPHSKRPD ILKTESLLPK
LDAALSGVGL PGCPKGPPSP GRSRRGKTRH RKASAKGSCG DLPGLRTAVP
PHEPGGPGSP GGLGGGPSAW EACPPALRGL HHDLLLRKMS SSSPDLLSAA
LGSRGRGATG GAGDPGSPPP ARGDTPPSEG SAPGSTSPDS PGGAKGEPPP
PVGPGEGVGL LGTGREGTSG RGGSRAGSQH LTPAALLYRA AVTRSQKRGI
SSEEEEGEVD SEVELTSSQR WPQSLNMRQS LSTFSSENPS DGEEGTASEP
SPSGTPEVGS TNTDERPDER SDDMCSQGSE IPLDPPPSEV IPGPEPSSLP
IPHQELLRER GPPNSEDSDC DSTELDNSNS VDALRPPASL PP
SEQ ID NO: 2
catalytic (kinase) domain amino acid sequence as defined in UniProt Q12852
ILD LQWVGSGAQG AVFLGRFHGE EVAVKKVRDL KETDIKHLRK
LKHPNIITFK GVCTQAPCYC ILMEFCAQGQ LYEVLRAGRP VTPSLLVDWS
MGIAGGMNYL HLHKIIHRDL KSPNMLITYD DVVKISDFGT SKELSDKSTK
MSFAGTVAWM APEVIRNEPV SEKVDIWSFG VVLWELLTGE IPYKDVDSSA
IIWGVGSNSL HLPVPSSCPD GFKILLRQCW NSKPRNRPSF RQILLHLDI
SEQ ID NO: 3
human DLK leucine zipper domain sequence
LSTPQE TYFKSQAEWR EEVKLHFEKI KSEGTCLHRL EEELVMRRRE
ELRHALDIRE HYERKLERAN NLYMELNALM LQLELKEREL LRREQALERR
CPGLLKPHPS RGLLHGNTME
SEQ ID NO: 4
Leucine-zipper 1 sequence of human DLK (as defined in Uniprot Q12852)
VKLHFEKIKSEGTCLHRLEEEL
SEQ ID NO: 5
Leucine-zipper 2 sequence of human DLK (as defined in Uniprot Q12852)
LNALMLQLELKERELLRREQAL
SEQ ID NO: 6
C-terminal deleted dnDLK with mutation at 302
MACLHETRTP SPSFGGFVST LSEASMRKLD PDTSDCTPEK DLTPTQCVLR
DVVPLGGQGG GGPSPSPGGE PPPEPFANSV LQLHEQDAGG PGGAAGSPES
RASRVRADEV RLQCQSGSGF LEGLFGCLRP VWTMIGKAYS TEHKQQQEDL
WEVPFEEILD LQWVGSGAQG AVFLGRFHGE EVAVKKVRDL KETDIKHLRK
LKHPNIITFK GVCTQAPCYC ILMEFCAQGQ LYEVLRAGRP VTPSLLVDWS
MGIAGGMNYL HLHKIIHRDL KSPNMLITYD DVVKISDEGT SKELSDKSTK
MAFAGTVAWM APEVIRNEPV SEKVDIWSFG VVLWELLTGE IPYKDVDSSA
IIWGVGSNSL HLPVPSSCPD GFKILLRQCW NSKPRNRPSF RQILLHLDIA
SADVLSTPQE TYFKSQAEWR EEVKLHFEKI KSEGTCLHRL EEELVMRRRE
ELRHALDIRE HYERKLERAN NLYMELNALM LQLELKEREL LRREQALERR
CPGLLKPHPS RGLLHGNTME
SEQ ID NO: 7
C-terminal deleted dnDLK with mutation at 302 and 43
MACLHETRTP SPSFGGFVST LSEASMRKLD PDTSDCTPEK DLEPTQCVLR
DVVPLGGQGG GGPSPSPGGE PPPEPFANSV LQLHEQDAGG PGGAAGSPES
RASRVRADEV RLQCQSGSGF LEGLFGCLRP VWTMIGKAYS TEHKQQQEDL
WEVPFEEILD LQWVGSGAQG AVFLGRFHGE EVAVKKVRDL KETDIKHLRK
LKHPNIITFK GVCTQAPCYC ILMEFCAQGQ LYEVLRAGRP VTPSLLVDWS
MGIAGGMNYL HLHKIIHRDL KSPNMLITYD DVVKISDFGT SKELSDKSTK
MAFAGTVAWM APEVIRNEPV SEKVDIWSFG VVLWELLTGE IPYKDVDSSA
IIWGVGSNSL HLPVPSSCPD GFKILLRQCW NSKPRNRPSF RQILLHLDIA
SADVLSTPQE TYFKSQAEWR EEVKLHFEKI KSEGTCLHRL EEELVMRRRE
ELRHALDIRE HYERKLERAN NLYMELNALM LQLELKEREL LRREQALERR
CPGLLKPHPS RGLLHGNTME
SEQ ID NO: 8
human MAP3K13 (leucine zipper kinase) leucine zipper domain amino
acid sequence
LATPQETYFKSQAEWREEVKKHFEKIKSEGTCIHRLDEELIRRRREELRHALDIREH
YERKLERANNLYMELSAIMLQLEMREKELIKREQAVEKKYPGTYKRHPVRPIIHPNA
ME
SEQ ID NO: 9
DLK full-length cDNA sequence encoding SEQ ID NO: 1
ATGGCTTGCCTCCATGAGACCCGAACACCCTCTCCTTCCTTTGGGGGCTTTGTGTCT
ACCCTAAGTGAGGCATCCATGCGCAAGCTGGACCCAGACACTTCTGACTGCACTCCC
GAGAAGGACCTGACGCCTACCCAGTGTGTACTTCGAGATGTGGTACCCCTTGGTGGG
CAGGGTGGGGGAGGGCCCAGCCCCTCCCCAGGTGGAGAGCCGCCCCCTGAGCCTTTT
GCCAACAGTGTCCTGCAGCTACATGAGCAGGATGCAGGGGGCCCAGGGGGAGCAGCT
GGGTCACCTGAGAGTCGGGCATCCAGAGTTCGAGCTGACGAGGTGCGACTGCAGTGC
CAGAGTGGCAGTGGCTTCCTTGAGGGCCTCTTTGGCTGCCTGCGCCCTGTCTGGACC
ATGATTGGCAAAGCCTACTCCACTGAGCACAAGCAGCAGCAGGAAGACCTTTGGGAG
GTCCCCTTTGAGGAAATCCTGGACCTGCAGTGGGTGGGCTCAGGGGCCCAGGGTGCT
GTCTTCCTGGGGCGCTTCCACGGGGAGGAGGTGGCTGTGAAGAAGGTGCGAGACCTC
AAAGAAWACCGACATCAAGCACTTGCGAAAGCTGAAGCACCCCAACATCATCACTTT
CAAGGGTGTGTGCACCCAGGCTCCCTGCTACTGCATCCTCATGGAGTTCTGCGCCCA
GGGCCAGCTGTATGAGGTACTGCGGGCTGGCCGCCCTGTCACCCCCTCCTTACTGGT
TGACTGGTCCATGGGCATCGCTGGTGGCATGAACTACCTGCACCTGCACAAGATTAT
CCACAGGGATCTCAAGTCACCCAACATGCTAATCACCTACGACGATGTGGTGAAGAT
CTCAGATTTTGGCACTTCCAAGGAGCTGAGTGACAAGAGCACCAAGATGTCCTTTGC
AGGGACAGTAGCCTGGATGGCCCCTGAGGTGATCCGCAATGAACCTGTGTCTGAGAA
GGTCGACATCTGGTCCTTTGGCGTGGTGCTATGGGAACTGCTGACTGGTGAGATCCC
CTACAAAGACGTAGATTCCTCAGCCATTATCTGGGGTGTGGGAAGCAACAGTCTCCA
TCTGCCCGTGCCCTCCAGTTGCCCAGATGGTTTCAAGATCCTGCTTCGCCAGTGCTG
GAATAGCAAACCACGAAATCGCCCATCATTCCGACAGATCCTGCTGCATCTGGACAT
TGCCTCAGCTGATGTACTCTCCACACCCCAGGAGACTTACTTTAAGTCCCAGGCAGA
GTGGCGGGAAGAAGTAAAACTGCACTTTGAAAAGATTAAGTCAGAAGGGACCTGTCT
GCACCGCCTAGAAGAGGAACTGGTGATGAGGAGGAGGGAGGAGCTCAGACACGCCCT
GGACATCAGGGAGCACTATGAAAGGAAGCTGGAGAGAGCCAACAACCTGTATATGGA
ACTTAATGCCCTCATGTTGCAGCTGGAACTCAAGGAGAGGGAGCTGCTCAGGCGAGA
GCAAGCTTTAGAGCGGAGGTGCCCAGGCCTGCTGAAGCCACACCCTTCCCGGGGCCT
CCTGCATGGAAACACAATGGAGAAGCTTATCAAGAAGAGGAATGTGCCACAGAAGCT
GTCACCCCATAGCAAAAGGCCAGATATCCTCAAGACGGAGTCTTTGCTCCCTAAACT
AGATGCAGCCCTGAGTGGGGTGGGGCTTCCTGGGTGTCCTAAGGGCCCCCCCTCACC
AGGACGGAGTCGCCGTGGCAAGACCCGTCACCGCAAGGCCAGCGCCAAGGGGAGCTG
TGGGGACCTGCCTGGGCTTCGTACAGCTGTGCCACCCCATGAACCTGGAGGACCAGG
AAGCCCAGGGGGCCTAGGAGGGGGACCCTCAGCCTGGGAGGCCTGCCCTCCCGCCCT
CCGTGGGCTTCATCATGACCTCCTGCTCCGCAAAATGTCTTCATCGTCCCCAGACCT
GCTGTCAGCAGCACTAGGGTCCCGGGGCCGGGGGGCCACAGGCGGAGCTGGGGATCC
TGGCTCACCACCTCCGGCCCGGGGTGACACCCCACCAAGTGAGGGCTCAGCCCCTGG
CTCCACCAGCCCAGATTCACCTGGGGGAGCCAAAGGGGAACCACCTCCTCCAGTAGG
GCCTGGTGAAGGTGTGGGGCTTCTGGGAACTGGAAGGGAAGGGACCTCAGGCCGGGG
AGGAAGCCGGGCTGGGTCCCAGCACTTGACCCCAGCTGCACTGCTGTACAGGGCTGC
CGTCACCCGAAGTCAGAAACGTGGCATCTCATCGGAAGAGGAGGAAGGAGAGGTAGA
CAGTGAAGTAGAGCTGACATCAAGCCAGAGGTGGCCTCAGAGCCTGAACATGCGCCA
GTCACTATCTACCTTCAGCTCAGAGAATCCATCAGATGGGGAGGAAGGCACAGCTAG
TGAACCTTCCCCCAGTGGCACACCTGAAGTTGGCAGCACCAACACTGATGAGCGGCC
AGATGAGCGGTCTGATGACATGTGCTCCCAGGGCTCAGAAATCCCACTGGACCCACC
TCCTTCAGAGGTCATCCCTGGCCCTGAACCCAGCTCCCTGCCCATTCCACACCAGGA
ACTTCTCAGAGAGCGGGGCCCTCCCAATTCTGAGGACTCAGACTGTGACAGCACTGA
ATTGGACAACTCCAACAGCGTTGATGCCTTGCGGCCCCCAGCTTCCCTCCCTCCATG
A
SEQ ID NO: 10
dnDLK S302A AC-terminus containing WPRE polynucleotide sequence
ATGGCTTGCCTCCATGAGACCCGAACACCCTCTCCTTCCTTTGGGGGCTTTGTGTCT
ACCCTAAGTGAGGCATCCATGCGCAAGCTGGACCCAGACACTTCTGACTGCACTCCC
GAGAAGGACCTGACGCCTACCCAGTGTGTACTTCGAGATGTGGTACCCCTTGGTGGG
CAGGGTGGGGGAGGGCCCAGCCCCTCCCCAGGTGGAGAGCCGCCCCCTGAGCCTTTT
GCCAACAGTGTCCTGCAGCTACATGAGCAGGATGCAGGGGGCCCAGGGGGAGCAGCT
GGGTCACCTGAGAGTCGGGCATCCAGAGTTCGAGCTGACGAGGTGCGACTGCAGTGC
CAGAGTGGCAGTGGCTTCCTTGAGGGCCTCTTTGGCTGCCTGCGCCCTGTCTGGACC
ATGATTGGCAAAGCCTACTCCACTGAGCACAAGCAGCAGCAGGAAGACCTTTGGGAG
GTCCCCTTTGAGGAAATCCTGGACCTGCAGTGGGTGGGCTCAGGGGCCCAGGGTGCT
GTCTTCCTGGGGCGCTTCCACGGGGAGGAGGTGGCTGTGAAGAAGGTGCGAGACCTC
AAAGAAACCGACATCAAGCACTTGCGAAAGCTGAAGCACCCCAACATCATCACTTTC
AAGGGTGTGTGCACCCAGGCTCCCTGCTACTGCATCCTCATGGAGTTCTGCGCCCAG
GGCCAGCTGTATGAGGTACTGCGGGCTGGCCGCCCTGTCACCCCCTCCTTACTGGTT
GACTGGTCCATGGGCATCGCTGGTGGCATGAACTACCTGCACCTGCACAAGATTATC
CACAGGGATCTCAAGTCACCCAACATGCTAATCACCTACGACGATGTGGTGAAGATC
TCAGATTTTGGCACTTCCAAGGAGCTGAGTGACAAGAGCACCAAGATGGCCTTTGCA
GGGACAGTAGCCTGGATGGCCCCTGAGGTGATCCGCAATGAACCTGTGTCTGAGAAG
GTCGACATCTGGTCCTTTGGCGTGGTGCTATGGGAACTGCTGACTGGTGAGATCCCC
TACAAAGACGTAGATTCCTCAGCCATTATCTGGGGTGTGGGAAGCAACAGTCTCCAT
CTGCCCGTGCCCTCCAGTTGCCCAGATGGTTTCAAGATCCTGCTTCGCCAGTGCTGG
AATAGCAAACCACGAAATCGCCCATCATTCCGACAGATCCTGCTGCATCTGGACATT
GCCTCAGCTGATGTACTCTCCACACCCCAGGAGACTTACTTTAAGTCCCAGGCAGAG
TGGCGGGAAGAAGTAAAACTGCACTTTGAAAAGATTAAGTCAGAAGGGACCTGTCTG
CACCGCCTAGAAGAGGAACTGGTGATGAGGAGGAGGGAGGAGCTCAGACACGCCCTG
GACATCAGGGAGCACTATGAAAGGAAGCTGGAGAGAGCCAACAACCTGTATATGGAA
CTTAATGCCCTCATGTTGCAGCTGGAACTCAAGGAGAGGGAGCTGCTCAGGCGAGAG
CAAGCTTTAGAGCGGAGGTGCCCAGGCCTGCTGAAGCCACACCCTTCCCGGGGCCTC
CTGCATGGAAACACAATGGAGTAG
SEQ ID NO: 11
dnDLK T43E S302A AC-terminus containing WPRE polynucleotide sequence
ATGGCTTGCCTCCATGAGACCCGAACACCCTCTCCTTCCTTTGGGGGCTTTGTGTCT
ACCCTAAGTGAGGCATCCATGCGCAAGCTGGACCCAGACACTTCTGACTGCACTCCC
GAGAAGGACCTGGAGCCTACCCAGTGTGTACTTCGAGATGTGGTACCCCTTGGTGGG
CAGGGTGGGGGAGGGCCCAGCCCCTCCCCAGGTGGAGAGCCGCCCCCTGAGCCTTTT
GCCAACAGTGTCCTGCAGCTACATGAGCAGGATGCAGGGGGCCCAGGGGGAGCAGCT
GGGTCACCTGAGAGTCGGGCATCCAGAGTTCGAGCTGACGAGGTGCGACTGCAGTGC
CAGAGTGGCAGTGGCTTCCTTGAGGGCCTCTTTGGCTGCCTGCGCCCTGTCTGGACC
ATGATTGGCAAAGCCTACTCCACTGAGCACAAGCAGCAGCAGGAAGACCTTTGGGAG
GTCCCCTTTGAGGAAATCCTGGACCTGCAGTGGGTGGGCTCAGGGGCCCAGGGTGCT
GTCTTCCTGGGGCGCTTCCACGGGGAGGAGGTGGCTGTGAAGAAGGTGCGAGACCTC
AAAGAAACCGACATCAAGCACTTGCGAAAGCTGAAGCACCCCAACATCATCACTTTC
AAGGGTGTGTGCACCCAGGCTCCCTGCTACTGCATCCTCATGGAGTTCTGCGCCCAG
GGCCAGCTGTATGAGGTACTGCGGGCTGGCCGCCCTGTCACCCCCTCCTTACTGGTT
GACTGGTCCATGGGCATCGCTGGTGGCATGAACTACCTGCACCTGCACAAGATTATC
CACAGGGATCTCAAGTCACCCAACATGCTAATCACCTACGACGATGTGGTGAAGATC
TCAGATTTTGGCACTTCCAAGGAGCTGAGTGACAAGAGCACCAAGATGGCCTTTGCA
GGGACAGTAGCCTGGATGGCCCCTGAGGTGATCCGCAATGAACCTGTGTCTGAGAAG
GTCGACATCTGGTCCTTTGGCGTGGTGCTATGGGAACTGCTGACTGGTGAGATCCCC
TACAAAGACGTAGATTCCTCAGCCATTATCTGGGGTGTGGGAAGCAACAGTCTCCAT
CTGCCCGTGCCCTCCAGTTGCCCAGATGGTTTCAAGATCCTGCTTCGCCAGTGCTGG
AATAGCAAACCACGAAATCGCCCATCATTCCGACAGATCCTGCTGCATCTGGACATT
GCCTCAGCTGATGTACTCTCCACACCCCAGGAGACTTACTTTAAGTCCCAGGCAGAG
TGGCGGGAAGAAGTAAAACTGCACTTTGAAAAGATTAAGTCAGAAGGGACCTGTCTG
CACCGCCTAGAAGAGGAACTGGTGATGAGGAGGAGGGAGGAGCTCAGACACGCCCTG
GACATCAGGGAGCACTATGAAAGGAAGCTGGAGAGAGCCAACAACCTGTATATGGAA
CTTAATGCCCTCATGTTGCAGCTGGAACTCAAGGAGAGGGAGCTGCTCAGGCGAGAG
CAAGCTTTAGAGCGGAGGTGCCCAGGCCTGCTGAAGCCACACCCTTCCCGGGGCCTC
CTGCATGGAAACACAATGGAGTAG
SEQ ID NO: 12
dominant negative LZK leucine zipper domain polynucleotide
sequence containing WPRE
ATGCTTGCCACCCCACAAGAAACTTACTTCAAGTCTCAGGCTGAATGGAGAGAAGAA
GTGAAAAAACATTTTGAGAAGATCAAAAGTGAAGGAACTTGTATACACCGGTTAGAT
GAAGAACTGATTCGAAGGCGCAGAGAAGAGCTCAGGCATGCGCTGGATATTCGTGAA
CACTATGAGCGGAAGCTTGAGCGGGCGAATAATTTATACATGGAATTGAGTGCCATC
ATGCTGCAGCTAGAAATGCGGGAGAAGGAGCTCATTAAGCGTGAGCAAGCAGTGGAA
AAGAAGTATCCTGGGACCTACAAACGACACCCTGTTCGTCCTATCATCCATCCCAAT
GCCATGGAGTGA
SEQ ID NO: 13
Human DLK polypeptide sequence, isoform 1, UniProt Q12852-1
MACTHETRTP SPSFGGFVST LSEASMRKLD PDTSDCTPEK DITPTHVLQL
HEQDAGGPGG AAGSPESRAS RVRADEVRLQ CQSGSGFLEG LFGCLRPVWT
MIGKAYSTEH KQQQEDLWEV PFEEILDLQW VGSGAQGAVF LGRFHGEEVA
VKKVRDLKET DIKHLRKLKH PNIITFKGVC TQAPCYCILM EFCAQGQLYE
VLRAGRPVTP SLLVDWSMGI AGGMNYLHLH KIIHRDLKSP NMLITYDDVV
KISDFGTSKE LSDKSTKMSF AGTVAWMAPE VIRNEPVSEK VDIWSFGVVL
WELLTGEIPY KDVDSSAIIW GVGSNSLHLP VPSSCPDGFK ILLRQCWNSK
PRNRPSFRQI LLHLDIASAD VLSTPQETYF KSQAEWREEV KLHFEKIKSE
GTCLHRLEEE LVMRRREELR HALDIREHYE RKLERANNLY MELNALMLQL
ELKERELLRR EQALERRCPG LLKPHPSRGL LHGNTMEKLI KKRNVPQKLS
PHSKRPDILK TESLLPKLDA ALSGVGLPGC PKGPPSPGRS RRGKTRHRKA
SAKGSCGDLP GLRTAVPPHE PGGPGSPGGL GGGPSAWEAC PPALRGLHHD
LLLRKMSSSS PDLLSAALGS RGRGATGGAG DPGSPPPARG DTPPSEGSAP
GSTSPDSPGG AKGEPPPPVG PGEGVGLLGT GREGTSGRGG SRAGSQHLTP
AALLYRAAVT RSQKRGISSE EEEGEVDSEV ELTSSQRWPQ SLNMRQSLST
FSSENPSDGE EGTASEPSPS GTPEVGSTNT DERPDERSDD MCSQGSEIPL
DPPPSEVIPG PEPSSLPIPH QELLRERGPP NSEDSDCDST ELDNSNSVDA
LRPPASLPP
SEQ ID NO: 14
WPRE nucleic acid sequence
ATCAACCTCTGGATTACAAAATTTGTGAAAGATTGACTGATATTCTTAACTATGT
TGCTCCTTTTACGCTGTGTGGATATGCTGCTTTAATGCCTCTGTATCATGCTATT
GCTTCCCGTACGGCTTTCGTTTTCTCCTCCTTGTATAAATCCTGGTTGCTGTCTC
TTTATGAGGAGTTGTGGCCCGTTGTCCGTCAACGTGGCGTGGTGTGCTCTGTGTT
TGCTGACGCAACCCCCACTGGCTGGGGCATTGCCACCACCTGTCAACTCCTTTCT
GGGACTTTCGCTTTCCCCCTCCCGATCGCCACGGCAGAACTCATCGCCGCCTGCC
TTGCCCGCTGCTGGACAGGGGCTAGGTTGCTGGGCACTGATAATTCCGTGGTGTT
GTCGGGGAAATCATCGTCCTTTCCTTGGCTGCTCGCCTGTGTTGCCAACTGGATC
CTGCGCGGGACGTCCTTCTGCTACGTCCCTTCGGCTCTCAATCCAGCGGACCTCC
CTTCCCGAGGCCTTCTGCCGGTTCTGCGGCCTCTCCCGCGTCTTCGCTTTCGGCC
TCCGACGAGTCGGATCTCCCTTTGGGCCGCCTCCCCGCCTG

What is claimed is:

1. A nucleic acid encoding a dominant negative Dual Leucine Zipper Kinase (dnDLK) polypeptide comprising an amino acid segment with a sequence having at least 80% identity to region 1-520 of SEQ ID NO:1, wherein the dnDLK polypeptide comprises at least one mutation, as determined with reference to SEQ ID NO:1, wherein the mutation is a substitution at position 302, wherein the substitution is any amino acid other than threonine.

2. The nucleic acid of claim 1, wherein the substitution at position 302 is S302A.

3. The nucleic acid of claim 1, wherein the dnDLK polypeptide is fewer than 550 amino acids in length.

4. The nucleic acid of claim 1, wherein the dnDLK polypeptide comprises a substitution at position 43.

5. The nucleic acid of claim 4, wherein the substitution at position 43 is E or D.

6. The nucleic acid of claim 5, wherein the substitution at position 43 is E.

7. The nucleic acid of claim 1, wherein the dnDLK polypeptide comprises a substitution at position 185.

8. The nucleic acid of claim 7, wherein the substitution at position 185 is K185A.

9. The nucleic acid of claim 1, wherein the dnDLK polypeptide comprising a substitution at position 424 or 426.

10. The nucleic acid of claim 9, wherein the dnDLK polypeptide further comprising a substitution at position 431, 438, 440, 445, 447, 486, 491, or 493.

11. The nucleic acid of claim 1, wherein the dnDLK polypeptide comprises the amino acid sequence of SEQ ID NO: 6 or SEQ ID NO:7.

12. The nucleic acid of claim 1, comprising a nucleic acid sequence having at least 95% identity to SEQ ID NO:11.

13. An isolated nucleic acid encoding a leucine zipper polypeptide that inhibits homodimerization and DLK heterodimerization with (LZK), wherein the polypeptide comprises an amino acid sequence having at least 80% identity to SEQ ID NO:8; or comprises SEQ ID NO:8.

14. The nucleic acid of claim 13, wherein the polypeptide is fewer than 150 amino acids in length.

15. An isolated nucleic acid encoding a dominant negative dual leucine zipper (dnDLK) polypeptide comprising an amino acid sequence having at least 90% identity to region 158-520 of SEQ ID NO:1, wherein the polypeptide comprises at least one mutation, as determined with reference to SEQ ID NO:1, wherein the at least one mutation is at position 302.

16. The nucleic acid of claim 15, wherein the dnDLK polypeptide comprises a substitution at position S302, wherein the substitution is any amino acid other than threonine.

17. The nucleic acid of claim 16, wherein the substitution is S302A.

18. The nucleic acid of any one of claims 15-17, wherein the dnDLK polypeptide comprises a substitution at position 185.

19. The nucleic acid of claim 18, wherein the substitution at position 185 is K185A.

20. The nucleic acid of claim 15, wherein the dnDLK polypeptide comprises a substitution at position 424 and/or 426.

21. The nucleic acid of claim 20, wherein the dnDLK polypeptide further comprises a substitution at position 431, 438, 440, 445, 447, 486, 491, or 493.

22. A vector comprising a nucleic acid of claim 1.

23. The vector of claim 22, wherein the vector is a viral vector.

24. The vector of claim 23, wherein the viral vector is an adeno-associated virus (AAV) vector.

25. The vector of claim 24, wherein the AAV vector is an AAV1, AAV2, AAV3, AAV4, AAV5, AAV6, AAV7, AAV8, AAV9, AAV10, AAV11-derived or pseudotyped AAV-derived vector.

26. The vector of claim 25, wherein the vector is AAV2.7m8.

27. The vector of claim 22, further comprising a Woodchuck Hepatitis Virus Posttranscriptional Regulatory Element (WPRE).

28. A host cell comprising a nucleic acid of claim 1 or a vector of claim 22.

29. The host cell of claim 28, wherein the host cell is a neuron.

30. The host cell of claim 29, wherein the neuron is a retinal ganglion.

31. The host cell of claim 29, wherein the host cell is a mammalian cell.

32. The host cell of claim 31, wherein the host cell is a human cell.

33. A method of inhibiting neuronal cell death, the method comprising introducing a nucleic acid of claim 1 or a vector of claim 22 into a neural cell.

34. The method of claim 33, wherein the neural cell is ex vivo.

35. The method of claim 33, wherein the neural cell is in vivo.

36. The method of claim 33, wherein the neural cell is an ophthalmic neuron.

37. The method of claim 36, wherein the ophthalmic neuron is a retinal ganglion.

38. The method of claim 33, wherein the neural cell is a photoreceptor cell.

39. The method of claim 33, wherein the neural cell is mammalian.

40. The method of claim 39, wherein the neuron is a human neural cell.

41. A method of treating or preventing neural cell death in a subject in need thereof, the method comprising administering a nucleic acid of claim 1 or a vector of claim 22 to the subject.

42. The method of claim 41, wherein the subject has glaucoma, age-related macular degeneration, choroidal neovascularization (CNV), myopia-associated CNV, diabetic retinopathy, macular oedema, and retinal vein occlusion.

43. The method of claim 41, wherein the subject has an inherited retinal disease.

44. The method of claim 43, wherein the inherited retinal disease is retinitis pigmentosa.

45. A polypeptide encoded by a nucleic acid of claim 1.
